SEQUENCE LISTING (58 sequences)

SEQ ID NO: 1 - protein, 12 aa, Hepatitis B virus
IPQSLDSWWTSL

SEQ ID NO: 2 - protein, 9 aa, Hepatitis B virus
GLSPTVWLS

SEQ ID NO: 3 - protein, 9 aa, Hepatitis B virus
FLLTRILTI

SEQ ID NO: 4 - protein, 9 aa, respiratory syncytial virus
SYIGSINNI

SEQ ID NO: 5 - protein, 15 aa, respiratory syncytial virus
ESYIGSINNITKQSA

SEQ ID NO: 6 - protein, 9 aa, Human papillomavirus type 16
RAHYNIVTF

SEQ ID NO: 7 - protein, 8 aa, respiratory syncytial virus
HWSISKPQ

SEQ ID NO: 8 - protein, 10 aa, Hepatitis C virus
YLLPRRGPRL

SEQ ID NO: 9 - protein, 9 aa, Hepatitis C virus
QLRRIDLLV

SEQ ID NO: 10 - protein, 9 aa, Hepatitis C virus
CINGVCWTV

SEQ ID NO: 11 - protein, 9 aa, Hepatitis C virus
SLMAFTAAV

SEQ ID NO: 12 - protein, 9 aa, Hepatitis C virus
ILAGYGAGV

SEQ ID NO: 13 - protein, 10 aa, Hepatitis C virus
RLIVFPDLGV

SEQ ID NO: 14 - protein, 10 aa, Hepatitis C virus
ADLMGYIPLV

SEQ ID NO: 15 - protein, 11 aa, Hepatitis C virus
SLLAPGAKQNV

SEQ ID NO: 16 - protein, 10 aa, Hepatitis C virus
KLVALGINAV

SEQ ID NO: 17 - protein, 10 aa, Hepatitis C virus
LLFNILGGWV

SEQ ID NO: 18 - protein, 9 aa, Hepatitis C virus
ILDSFDPLV

SEQ ID NO: 19 - protein, 9 aa, Hepatitis B virus
GLQDCTMLV

SEQ ID NO: 20 - protein, 9 aa, Hepatitis B virus
VLQAGFFLL

SEQ ID NO: 21 - protein, 9 aa, Hepatitis B virus
FLGGTTVCL

SEQ ID NO: 22 - protein, 9 aa, Hepatitis B virus
LLDYQGMLP

SEQ ID NO: 23 - protein, 9 aa, EPSTEIN BARR VIRUS
FLYALALLL

SEQ ID NO: 24 - protein, 9 aa, CYTOMEGALOVIRUS
NLVPWWRTV

SEQ ID NO: 25 - protein, 8 aa, Homo sapiens
SINNFEKL

SEQ ID NO: 26 - protein, 9 aa, Human immunodeficiency virus
ILKEPVHGV

SEQ ID NO: 27 - protein, 10 aa, Hepatitis B virus
SILSPFLPLL

SEQ ID NO: 28 - protein, 9 aa, Cytomegalovirus
TMYGGISLL

SEQ ID NO: 29 - protein, 10 aa, Human Metapneumovirus
KLILALLTFL

SEQ ID NO: 30 - protein, 9 aa, Murine Metapneumovirus
VGALIFTKL

SEQ ID NO: 31 - protein, 226 aa, Hepatitis B Virus
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 32 - protein, 237 aa, Hepatitis B Virus
MENITSGFLGPLLRTTRILTILLAESYIGSINNITKQSAALANSGGQNSQSPTSNHSPTSCPPTCPGYRWMCLRRFIIFLFILLLCLIFLLVLKCPLIPGSSTTSTGPCRTCMTTGHWSISKPQTGQGTSMYPSCCCTKPSDGNCTCIPIPSSWAFGKFLWEWASARFSWLSLLVPFVQWFVGLSARAHYNIVTFALSVIWMMWYWGPSLYPRAASIINFEKLALPRPIFFCLWVYI

SEQ ID NO: 33 - protein, 253 aa, Hepatitis B Virus
MENITSGFLGPLLRTTRILTILLAESYIGSINNITKQSAALANSGAARAHYNIVTFALSGGQNSQSPTSNHSPTSCPPTCPGYRWMCLRRFIIFLFILLLCLIFLLVLKCPLIPGSSTTSTGPCRTCMTTGHWSISKPQTGQGTSMYPSCCCTKPSDGNCTCIPIPSSWAFGKFLWEWASARFSWLSLLVPFVQWFVGLSARAHYNIVTFALSVIWMMWYWGPSLYPRAARAHYNIVTFALPRPIFFCLWVYI

SEQ ID NO: 34 - protein, 262 aa, Hepatitis B Virus
MENITSGFLGPLLRTAARAHYNIVTFALRTTRILTILLARAHYNIVTFALANSGAARAHYNIVTFALSGGQNSQSPTSNHSPTSCPPTCPGYRWMCLRRFIIFLFILLLCLIFLLVLKCPLIPGSSTTSTGPCRTCMTTGHWSISKPQTGQGTSMYPSCCCTKPSDGNCTCIPIPSSWAFGKFLWEWASARFSWLSLLVPFVQWFVGLSARAHYNIVTFALSVIWMMWYWGPSLYPRAARAHYNIVTFALPRPIFFCLWVYI

SEQ ID NO: 35 - protein, 282 aa, Hepatitis B Virus
MENITSGFLGPLLRTAASLYNTVATLALRTTRILTILLAESYIGSINNITKQSAALANSGAAFLYALALLLALSGGQNSQSPTSNHSPTSCPPTCPGYRWMCLRRFIIFLFILLLCLIFLLVLKAANLVPMVATVALLKCPLIPGSSTTSTGPCRTCMTTGHWSISKPQTGQGTSMYPSCCCTKPSDGNCTCIPIPSSWAFGKFLWEWASARFSWLSLLVPFVQWFVGLSARAHYNIVTFALSVIWMMWYWGPSLYPRAASIINFEKLALPRPIFFCLWVYI

SEQ ID NO: 36 - protein, 282 aa, Hepatitis B Virus
MENITSGFLGPLLRTAAILKEPVHGVALRTTRILTILLAESYIGSINNITKQSAALANSGAAFLYALALLLALSGGQNSQSPTSNHSPTSCPPTCPGYRWMCLRRFIIFLFILLLCLIFLLVLKAATMYGGISLLALLKCPLIPGSSTTSTGPCRTCMTTGHWSISKPQTGQGTSMYPSCCCTKPSDGNCTCIPIPSSWAFGKFLWEWASARFSWLSLLVPFVQWFVGLSARAHYNIVTFALSVIWMMWYWGPSLYPRAASIINFEKLALPRPIFFCLWVYI

SEQ ID NO: 37 - protein, 219 aa, Hepatitis B Virus
MENITSGFLGPLLVLQAGFFLLTRILTILLANFLGGTTVCLGQNSQSPTSNHSPTSCPPTCPGYRWMCLRRFIIFLFILLLCLIFLLVLLDYQGMLPVCPLIPGSSTTSTGPCRTCMTTQGTSMYPSCCCTKPSDGNCTCIPIPSSWAFGKFLWEWASARFSWLSLLVPFVQWFVGLSSVIWMMWYWGPSLYPRAARAHYNIVTFALPRPIFFCLWVYI

SEQ ID NO: 38 - protein, 262 aa, Hepatitis B Virus
MENITSGFLGPLLVLQAGFFLLTRILTILLAESYIGSINNITKQSAALANFLGGTTVCLGQNSQSPTSNHSPTSCPPTCPGYRWMCLRRFIIFLFILLLCLIFLLVLLDYQGMLPVCPLIPGSSTTSTGPCRTCMTTGHWSISKPQTGQGTSMYPSCCCTKPSDGNCTCIPIPSSWAFGKFLWEWASARFSWLSLLVPFVQWFVGLSAAKLILALLTFLALSVIWMMWYWGPSLYPRAAVGALIFTKLALPRPIFFCLWVYI

SEQ ID NO: 39 - protein, 226 aa, Hepatitis B Virus
MENITSGFLGPLLVLQAGFFLLTRILTESYIGSINNITKQSAGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVRAHYNIVTFVIWMMWYWGPSLYSILSPFLPLLPIFFCLWVYI

SEQ ID NO: 40 - DNA, 741 bp, Hepatitis B Virus
atggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaattatgggttatgtcattggatgttatgggtccttgccacaagaacacatcatacaaaaaa

SEQ ID NO: 41 - DNA, 714 bp, Hepatitis B Virus
atggagaacatcacatcaggattcctaggaccccttctccgtacgacaagaatcctcacaatactgctagcggagtcctatatcggctccatcaacaacatcaccaaacaaagtgctgcgctagcgaattccggaggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttaagtgtcctctaattccaggatcctcaacaaccagcacgggaccatgccggacctgcatgactaccggtcactggagtatcagtaaaccccagaccggtcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggcttagcgccagggcccactacaacatcgtgaccttcgcgcttagcgttatatggatgatgtggtattgggggccaagtctgtatccgcgggccgccagcatcatcaacttcgagaagctggccctgccgcggccaattttcttttgtctttgggtatacatttaa

SEQ ID NO: 42 - DNA, 762 bp, Hepatitis B Virus
atggagaacatcacatcaggattcctaggaccccttctccgtacgacaagaatcctcacaatactgctagcggagtcctatatcggctccatcaacaacatcaccaaacaaagtgctgcgctagcgaattccggagccgccagggcccactacaacatcgtgaccttcgccctgtccggaggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttaagtgtcctctaattccaggatcctcaacaaccagcacgggaccatgccggacctgcatgactaccggtcactggagtatcagtaaaccccagaccggtcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggcttagcgccagggcccactacaacatcgtgaccttcgcgcttagcgttatatggatgatgtggtatctgccgcggccaattttcttttgtctttgggtatacatttaatgggggccaagtctgtatccgcgggccgccagggcccactacaacatcgtgaccttcgcc

SEQ ID NO: 43 - DNA, 789 bp, Hepatitis B Virus
atggagaacatcacatcaggattcctaggaccccttctccgtacggccgccagggcccactacaacatcgtgaccttcgccctgcgtacgacaagaatcctcacaatactgctagcgagggcccactacaacatcgtgaccttcgcgctagcgaattccggagccgccagggcccactacaacatcgtgaccttcgccctgtccggaggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttaagtgtcctctaattccaggatcctcaacaaccagcacgggaccatgccggacctgcatgactaccggtcactggagtatcagtaaaccccagaccggtcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggcttagcgccagggcccactacaacatcgtgaccttcgcgcttagcgttatatggatgatgtggtattgggggccaagtctgtatccgcgggccgccagggcccactacaacatcgtgaccttcgccctgccgcggccaattttcttttgtctttgggtatacatttaa

SEQ ID NO: 44 - DNA, 847 bp, Hepatitis B Virus
atggagaacatcacatcaggattcctaggaccccttctccgtacggccgccagcctgtacaacaccgtggccaccctggccctgcgtacgacaagaatcctcacaatactgctagcggagtcctatatcggctccatcaacaacatcaccaaacaaagtgctgcgctagcgaattccggagccgccttcctgtacgccctggccctgctgctggccctgtccggaggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttaaggccgccaacctggtgcccatggtggccaccgtggccctgcttaagtgtcctctaattccaggatcctcaacaaccagcacgggaccatgccggacctgcatgactaccggtcactggagtatcagtaaaccccagaccggtcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggcttagcgccagggcccactacaacatcgtgaccttcgcgcttagcgttatatggatgatgtggtattgggggccaagtctgtatccgcgggccgccagcatcatcaacttcgagaagctggccctgccgcggccaattttcttttgtctttgggtatacattt

SEQ ID NO: 45 - DNA, 847 bp, Hepatitis B Virus
atggagaacatcacatcaggattcctaggaccccttctccgtacggccgccatcctgaaggagcctgtgcacggcgtcgccctgcgtacgacaagaatcctcacaatactgctagcggagtcctatatcggctccatcaacaacatcaccaaacaaagtgctgcgctagcgaattccggagccgccttcctgtacgccctggccctgctgctggccctgtccggaggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttaaggccgccaccatgtacggcggaatcagcctgctggccctgcttaagtgtcctctaattccaggatcctcaacaaccagcacgggaccatgccggacctgcatgactaccggtcactggagtatcagtaaaccccagaccggtcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggcttagcgccagggcccactacaacatcgtgaccttcgcgcttagcgttatatggatgatgtggtattgggggccaagtctgtatccgcgggccgccagcatcatcaacttcgagaagctggccctgccgcggccaattttcttttgtctttgggtatacattt

SEQ ID NO: 46 - DNA, 660 bp, Hepatitis B Virus
atggagaacatcacatcaggattcctaggaccccttctcgtgttacaggcggggtttttcttgttgacaagaatcctcacaatactgctagcgaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactacccaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggcttagcagcgttatatggatgatgtggtattgggggccaagtctgtatccgcgggccgccagggcccactacaacatcgtgaccttcgccctgccgcggccaattttcttttgtctttgggtatacatttaa

SEQ ID NO: 47 - DNA, 789 bp, Hepatitis B Virus
atggagaacatcacatcaggattcctaggaccccttctcgtgttacaggcggggtttttcttgttgacaagaatcctcacaatactgctagcggagtcctatatcggctccatcaacaacatcaccaaacaaagtgctgcgctagcgaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactaccggtcactggagtatcagtaaaccccagaccggtcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggcttagcgccgccaagctgatcctggccctgctgaccttcctggcgcttagcgttatatggatgatgtggtattgggggccaagtctgtatccgcgggccgccgtgggcgccctgatcttcaccaagctggccctgccgcggccaattttcttttgtctttgggtatacatttaa

SEQ ID NO: 48 - DNA, 679 bp, Hepatitis B Virus
atggagaacatcacatcaggattcctaggaccccttctcgtgttacaggcggggtttttcttgttgacaagaatcctcacagagtcctatatcggctccatcaacaacatcaccaaacaaagtgctgggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtaagggcccactacaacatcgtgaccttcgttatatggatgatgtggtattgggggccaagtctgtacagcatcttgagtccctttttaccgctgttaccaattttcttttgtctttgggtatacattt

SEQ ID NO: 49 - protein, 9 aa, Respiratory Synctial Virus
NPKASLLSL

SEQ ID NO: 50 - protein, 9 aa, respiratory syncytial virus
AELDRTEEY

SEQ ID NO: 51 - protein, 10 aa, respiratory syncytial virus
RELPRFMNYT

SEQ ID NO: 52 - protein, 9 aa, respiratory syncytial virus
IAVGLLLYC

SEQ ID NO: 53 - protein, 12 aa, respiratory syncytial virus
ARAHYNIVTFAL

SEQ ID NO: 54 - protein, 13 aa, respiratory syncytial virus
AARAHYNIVTFAL

SEQ ID NO: 55 - protein, 12 aa, respiratory syncytial virus
AARAHYNIVTFA

SEQ ID NO: 56 - protein, 9 aa, Hepatitis B virus
SLYNTVATL

SEQ ID NO: 57 - protein, 9 aa, Hepatitis B Virus
SILSPFLPL

SEQ ID NO: 58 - protein, 9 aa, Hepatitis B Virus
IPQSLDSWW

(19) United States
(12) Patent Application Publication
(10) Pub. No.: US 2009/0220537 A1
(43) Pub. Date: Sep. 3, 2009

(54) VACCINE DELIVERY SYSTEM

(75) Inventors: Robert Tindle, Kenmore (AU); Hans Netter, Clayton (AU); Wai Ping Yvonne Woo, Eight Mile Plains (AU)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: THE UNIVERSITY OF QUEENSLAND, Brisbane, Queensland (AU)

(21) Appl. No.: 11/911,442

(22) PCT Filed: Apr. 12, 2006
(86) PCT No.: PCT/AU06/00491

§ 371 Date: Jun. 6, 2008

Related U.S. Application Data
(60) Provisional application No. 60/670,475, filed on Apr. 12, 2005.

(51) Int. Cl. A61K 39/21; C07K 14/005; C12N 15/11; C12N 15/00; C12N 5/00; C12N 5/08; C12N 1/19; C12P 21/02; C12N 7/00; A61K 39/12; A61K 39/29

(52) U.S. Cl. 424/188.1; 530/326; 536/23.72; 435/320.1; 435/325; 435/366; 435/254.2; 435/69.1; 435/235.1; 424/186.1; 424/189.1

(57) ABSTRACT

An isolated protein comprising a hepatitis B surface antigen (HBsAg) amino acid sequence, and encoding nucleic acid, are provided wherein one or more immunogenic T cell epitopes of the HBsAg are respectively substituted with one of more immunogenic T cell epitopes of a protein other than HBsAg. Typically, the T cell epitopes are of a pathogen or tumour protein. The isolated protein may have endogenous HBsAg epitopes substituted with multiple copies of the same epitope or with different HBsAg epitopes. B cell epitopes may also be present. Also provided are expression constructs, VLPs, compositions, vaccines and methods of treatment that may be useful in the prophylactic and/or therapeutic treatment of diseases including human papillomavirus, respiratory syncytial virus, human immunodeficiency virus (HIV), cytomegalovirus (CMV), Epstein Barr virus (EBV), rotavirus, hepatitis B virus, parainfluenza virus, hepatitis C virus, Plasmodium falciparum, influenza virus, Mycobacterium tuberculosis measles virus and human metapneumovirus.

### FIELD OF THE INVENTION

THIS INVENTION relates to an immunogenic protein and encoding nucleic acid for use in a vaccine delivery system. More particularly, this invention relates to a recombinant vaccine that induces protective T cell immunity to viral and/or microbial infections and cancer.

### BACKGROUND OF THE INVENTION

Infectious diseases remain the worlds leading cause of death, accounting for at least 33% of the people who die each year. In 2002 more than 90% of the deaths from infectious diseases were caused by only a handful of diseases such as AIDS, respiratory syncytial virus (RSV), tuberculosis, malaria, measles and rotavirus.

Most of the world's infectious diseases and some of the world's cancers can be prevented by vaccination. However, vaccine strategies capable of safely and effectively inducing cytotoxic T lymphocyte (CTL) responses which are acceptable in humans have not proven easy to develop (Gupta, R. K. and Siber, G. R. 1995. Vaccine 13, 1263-1276).

There are no effective human vaccines for the prophylactic or therapeutic treatment of diseases such as AIDS and RSV. AIDS is a leading cause of death especially in regions such as Sub-Saharan Africa and Asia. RSV causes lower respiratory tract infection in infants worldwide, and is a significant cause of morbidity and mortality in the elderly.

In addition there is no effective vaccine or vaccine vector that can simultaneously deliver protection and/or therapy against a plurality of the above diseases.

Viral capsid or envelope proteins self-assemble into virus-like particles (VLPs) which generally induce a more potent immune response when used for vaccination than denatured or soluble proteins. This is likely to be due to the particulate nature of VLPs and repeated subunit structure. Antigens from a number of infectious pathogens may be synthesised as VLPs in heterologous expression systems. VLPs have a number of advantages over conventional vaccines: (i) VLP vaccines mimic the immunogenicity of live attenuated vaccines; (ii) they can induce a CTL and B cell response in the absence of adjuvant; (iii) VLPs cannot replicate and are therefore non-infectious; (iv) they can be produced in large quantities; and (v) are easily enriched and purified.

The small envelope protein of hepatitis B surface virus (HBsAg-S) expressed by the hepatitis B virus (HBV) can self assemble into highly organised VLPs. HBsAg VLPs consist of 100-150 subunits of the 226 residue p24 HBsAg-S protein. The HBsAg molecule contains a 70 amino acid hydrophilic external domain flanked by 57 amino acid cytosolic domain and a 56 amino acid hydrophobic carboxy terminus. The external domain contains a highly immunogenic double-looped tertiary structure containing the major B-epitopes of the protein (the ‘a’-determinant). Prophylactic immunisation with HBV vaccine composed of recombinant HBsAg-S results in protective immunity due to an antibody response directed predominantly to the ‘a’-determinant region. HBsAg particles are also extremely efficient at inducing long-lived cytotoxic T cell (CTL) responses via an alternative endosomal/lysosomal processing pathway for MHC class 1 presentation (Bohm, et al. 1995 J. Immunol. 155, 3313-3321), comparing favourably with traditionally strong CTL inducers (Schirmbeck, et al. 1994J. Immunol. 152, 1110-1119).

In acutely HBV infected patients, CD8+ T cell responses are specific for a number of epitopes derived from HBsAg protein, and are associated with viral clearance (Nayersina, et al. 1993 J. Immunol. 150: 4659-4671; Rehermann, et al. 1996 Nat. Med. 2:1104-1108). HBsAg may also be delivered as a DNA vaccine, where CTL induction by intracellular translated HBsAg protein occurs through a ‘classical’ endogenous pathway as well as through an ‘alternative’ pathway via secreted HBsAg particles (Schirmbeck, et al. 1995. J. Virol. 69: 5929-5934). Genetic immunisation of HLA transgenic mice with wild-type HBsAg DNA elicited simultaneous CTL responses against a number of ‘human’ HBsAg epitopes (Loirat, et al. 2000 J. Immunol. 165: 4748-4755; Vaccine 20: 3137-3147).

Synthesis of hybrid HBsAg molecules, in which foreign epitopes are inserted into the HBsAg molecule has been described (Delpeyroux, et al. 1988 J. Virol. 62, 1836-1839; Bryder, et al. 1999 Cell Biol. 18, 219-225). While mutational analysis of HBsAg particle assembly has revealed insertions of foreign sequences into the luminal (Delpeyroux, et al. 1987 J. Mol. Biol. 195, 343-350; Bruss & Ganem 1991 J. Virol. 65, 3813-3820) or the cytoplasmic-loop (Delpeyroux, et al. 1987 J. Mol. Biol. 195, 343-350), the stability and secretion competence of the resultant recombinant VLPs is highly variable.

Reference is also made to US Patent Application No. 2003/0211996 which describes an isolated polynucleotide comprising a HBsAg-S coding sequence that is adapted to receive a heterologous protein coding sequence within an external loop of HBsAg. The heterologous protein coding sequence is inserted into the HBsAg coding sequence at the location of restriction sites in the HBsAg coding sequence. US Patent Application No. 2004/0146529 describes hepatitis B and hepatitis C chimeric antigens which form VLPs when co-expressed with HBsAg. The chimeric antigen comprises an immunogenic peptide derived from a hepatitis C protein coupled or attached to the amino terminus of HBsAg.

Prange et al. (1995, J. General Virology, 76, 2131-2140) describes a hepatitis B chimeric antigen with an amino terminal sequence of a large HBsAg antigen coupled or attached to the carboxy terminus of HBsAg-S.

### SUMMARY OF THE INVENTION

The inventors have developed a generic vaccine delivery system that can reliably evoke a T cell immune response in an animal, to one or more of a plurality of different pathogens. The invention is therefore broadly directed to a vaccine delivery system using hepatitis B surface antigen (HBsAg) as a generic vector for delivery of immunogenic epitopes derived from a single pathogenic origin or from a plurality of pathogens.

In one particularly broad form, the invention is broadly directed to a vaccine delivery system using HBsAg for delivery of immunogenic epitopes derived from respiratory syncytial virus.

In a first aspect, the invention provides an isolated protein comprising a HBsAg amino acid sequence wherein one or more immunogenic epitopes of the HBsAg are respectively substituted by one or more immunogenic epitopes of a protein other than HBsAg.

Preferably, the one or more immunogenic epitopes of the protein other than HBsAg is a T cell epitope.

Preferably, the T cell epitope is a CTL epitope.

In particular embodiments, the one or more immunogenic epitopes of the HBsAg that are substituted are selected from the group consisting of IPQ, GLS, FLL, VLQ, FLG, LLD, and SIL, but not limited thereto.

Suitably, said protein other than HBsAg is derived from a pathogen or is a tumour associated antigen.

In one embodiment, the isolated protein comprises one or more immunogenic epitopes of a single protein other than HBsAg.

In another embodiment, the isolated protein comprises one or more immunogenic epitopes of a plurality of proteins other than HBsAg.

Preferably, the CTL epitope of a protein other than HBsAg is selected from the group consisting of NPKASLLSL, AELDRTEEY, RELPRFMNYT, IAVGLLLYC, ESYIGSINNITKQSA or RAHYNIVTF.

In yet another embodiment, the isolated protein further comprises a B cell epitope of a protein other than HBsAg. Preferably, the B cell epitope is a mimotope with the sequence of HWSISKPQ. More preferably, the B cell epitope of a protein other than HBsAg is inserted within the a-determinant region of HBsAg.

It will therefore be appreciated that the plurality of proteins other than HBsAg may be derived or obtainable from one or a plurality of different pathogens.

Preferably, said pathogen is selected from the group consisting of bacteria, parasites, viruses, protozoa and other infectious agents.

Suitably, said pathogen is selected from the group consisting of human papillomavirus, respiratory syncytial virus, human immunodeficiency virus (HIV), cytomegalovirus (CMV), Epstein Barr virus (EBV), rotavirus, hepatitis B virus, parainfluenza virus, hepatitis C virus, Plasmodium falciparum, influenza virus, Mycobacterium tuberculosis measles virus and human metapneumovirus. Preferably, the pathogen is respiratory syncytial virus or human papillomavirus.

It will therefore be appreciated that the plurality of proteins other than HBsAg may be one or more tumour associated antigens.

It will also be appreciated that the invention contemplates substitution with corresponding epitopes of hepatitis B virus strains having variant sequences.

In a second aspect, the invention provides an isolated nucleic acid encoding the isolated protein of the first aspect. Suitably although not limited thereto, said isolated nucleic acid is DNA.

In a third aspect, the invention provides an expression construct comprising an isolated nucleic acid according to the second aspect operably-linked or connected to one or more regulatory sequences in an expression vector.

In a fourth aspect, the invention provides a host cell comprising the expression construct of the third aspect.

In a particular embodiment, the host cell is a cell capable of producing secretion-competent virus-like particles (VLPs).

Preferably, the host cell is of eukaryotic origin.

In another embodiment, VLPs may be purified from a mammalian cell line. Preferably, VLPs are purified from HuH-7 cells.

In another embodiment, VLPs can be expressed and purified from yeast cells.

In a fifth aspect, the invention provides a VLP comprising a plurality of isolated proteins of the first aspect.

In a sixth aspect, the invention provides a VLP comprising the isolated nucleic acid of the second aspect.

In a seventh aspect, the invention provides a method for producing an isolated nucleic acid including the step of substituting each of one or more nucleotide sequences of an isolated nucleic acid encoding one or more HBsAg immunogenic epitopes with a nucleotide sequence encoding one or more immunogenic epitopes of a protein other than HBsAg.

In particular embodiments, the HBsAg immunogenic epitopes are selected from the group consisting of IPQ, GLS, FLL, VLQ, FLG, LLD, and SIL, but not limited thereto.

In a eighth aspect, the invention provides a method of producing a VLP comprising the steps of:
(i) introducing the isolated nucleic acid of the second aspect into a packaging cell which is capable of a producing a VLP;
(ii) culturing said packaging cell under conditions that facilitate production of the VLP; and
(iii) isolating the VLP.

In a ninth aspect, the invention provides a pharmaceutical composition comprising an isolated protein, isolated nucleic acid or VLP according to any of the aforementioned aspects and a pharmaceutically acceptable carrier, diluent or excipient.

Preferably, the pharmaceutical composition of the invention is an immunotherapeutic composition.

More preferably, the pharmaceutical composition is a vaccine.

Preferably, the pharmaceutical composition is particularly useful for treating diseases, disorders and conditions which depend upon a cytotoxic lymphocyte and/or Th1 antibody response.

Compositions according to this aspect may be used either prophylactically or therapeutically.

In a tenth aspect, the invention provides a method of treating an animal to thereby modulate an immune response in said animal to prophylactically or therapeutically treat a disease, disorder or condition. It will be appreciated that the disease, disorder or condition can be caused by any infectious organism or neoplastic condition for which a disease-specific or highly disease-associated antigen is, or will be known.

Preferably, the disease, disorder or condition is caused by pathogens selected from the group consisting human immunodeficiency virus (HIV), hepatitis B virus, human papillomavirus, rotavirus, RSV, cytomegalovirus (CMV), Epstein Barr virus (EBV), Mycobacterium tuberculosis, hepatitis C virus, Plasmodium falciparum, influenza virus, measles virus, parainfluenza virus and human metapneumovirus.

The invention further contemplates treatment of a disease, disorder or condition that is cancer. Preferably, the cancer is selected from the group consisting of melanoma or hepatocellular carcinoma.

In an eleventh aspect, the invention provides a method of immunizing an animal including the step of administering the pharmaceutical composition of the eighth aspect to said animal to induce immunity in said animal.

An animal can be selected from the group consisting of humans, domestic livestock, laboratory animals, companion animals, poultry and other animals of commercial importance, although without limitation thereto.

Preferably, the animal is a mammal.

More preferably, the animal is a human.

Immunity may be antibody-mediated and/or cell mediated immunity such as T cell mediated immunity.

In one embodiment, T cell immunity is characterized by a CD8+ cytotoxic T lymphocyte (CTL) response.

Preferably, T cell immunity is characterized by induction of a long-term effector CD8+ CTL response.

In another embodiment, T cell immunity is characterized by a CD4+ T cell response.

In one particular embodiment, the method of immunization induces immunity to viral infection.

In another particular embodiment, the method of immunization induces immunity to cancers such as cervical cancer.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE FIGURES AND TABLES

Table 1. The sequences of the HBsAg, RSV and HPV epitopes.

Table 2. Summary of constructs in which endogenous HBsAg CTL epitopes are replaced with foreign CTL epitopes.¹ The net size change incurred by CTL epitope replacement is determined by the number of amino acids of the endogenous epitope deleted, the number of amino acids introduced by in-frame insertion of the RE site, and the number of amino acids in the foreign epitope that is inserted.² The net size change increases by a further 8 amino acids of the inserted B-mimotope which was inserted into a sequence located in the hydrophilic loop of HBsAg.

Table 3. RSV infection in lungs of mice after immunization with rHBsAg encoding a protective CTL epitope of RSV M2 protein or control. Immunised mice were challenged intranasally with 8×10⁵ pfu of RSV, 3 weeks after immunization. RSV was quantified in the lungs 4 days later. Limit of detection=5×10² pfu/lung. *p<0.0001 (Groups 1, 2 vs Group 4); **p<0.0001 (Group 1, 2 vs Group 4) (ANOVA).

Table 4. HLA A2.1-restricted HCV CTL epitopes shown to induce strong CTL-responses and associated with acute and self-limiting hepatitis C.

Table 5. Amino acid sequence information of endogenous HBsAg and foreign epitopes for constructs used throughout this document.

FIG. 1. Schema for derivation of recombinant HBsAg plasmid DNA encoding a foreign CTL epitope. PCR-driven site-directed mutagenesis was performed on the HBsAg-S gene with primers designed to delete an endogenous HBsAg CTL epitope (in this case GLS (Table 1)) and insert a unique restriction enzyme site (in this case BlpI). Annealed oligonucleotides encoding a foreign CTL epitope (in this case RSVM₁₅) were cloned into the BlpI-digested plasmid to derive HBsAg recombinant for the foreign epitope (details in Materials and Methods).

FIG. 2. Secretion of rHBsAg protein by cells transfected with recombinant HBsAg DNA. (A) Detection of HBsAg protein in cell culture fluid. HuH-7 cells were cotransfected with plasmids encoding recombinant or wild-type HBsAg proteins, and pSEAP. Supernatants were harvested and HBsAg measured by a Abbott Prism HBsAg chemiluminescence assay. The light counts were normalised by a SEAP assay (B, C). Detection of hepatitis delta virus large antigen (HDAg-L) in the presence of different recombinant HBsAg proteins. HuH-7 cells were cotransfected with plasmids encoding recombinant or wild-type HBsAg proteins, and a plasmid encoding HDAg-L. Cell culture supernatant (10 mls) was pelleted through a sucrose cushion, resuspended in sample buffer, and the supernatants (B) and cell pellets (C) analysed by immunoblot specific for HDAg. Lane 1, pM2₉ΔIPQ. Lane 2, pM2₁₅ΔIPQ. Lane 3, pM2₁₅ΔGLS. Lane 4, pHBsAg wild-type. Lane 5 pHBsAg wild type without pHDAg-L. Lane 6, pHDAg-L without pHBsAg wild type Lane 7, neither pHDAg-L or pHBsAg.

FIG. 3. Immunisation with rHBsAg DNA encoding a RSV CTL epitope at the ΔIPQ site evokes RSV-directed effector and memory CTL responses.

(A). Epitope-specific IFN-γ-secreting T-cell response following immunisation with recombinant HBsAg plasmid DNA encoding a RSV CTL epitope. H-2^{d} mice (BALB/c, 3 per group) were immunised twice intradermally (id) with rHBsAg DNA encoding the M2 9-mer (pM2₉ΔIPQ) or the M2 15-mer (pM2₁₅ΔIPQ) RSV CTL epitope at the site of deleted IPQ HBsAg CTL epitope, as indicated. Control mice were immunised twice with wild-type HBsAg DNA (pHBsAg.) id, or once with M2₁₅ or IPQ peptide plus adjuvant intramuscularly (im). IFN-γ-secreting cells were quantified by ELISPOT assay in splenocytes harvested at 14 days (DNA immunisations) or 10 days (peptide immunisations) after immunisation, and incubated for 15-18 h with specific peptide (M2₁₅ or IPQ), or without peptide as shown. Histogram bars represent means+/−standard deviation of 3 replicates. (B) Epitope specific CTL memory responses following immunisation with recombinant pHBsAg DNA encoding a RSV CTL epitope. Groups of mice (3 per group) were immunised as above. Percent cytotoxicity of splenocytes restimulated with M2₁₅ or IPQ peptide was measured in a ⁵¹Cr release assay using P815 target cells pulsed with M2₁₅ or IPQ peptide or without peptide as shown. Data points represent means of 3 replicates+/−standard deviation. (Note in some cases standard deviation bars, though plotted are too small to appear). For ELISPOT, IFN-γ secretion with and without peptide was compared by Student's t-test. For CTL assay, cytotoxicity against targets with and without peptide at an E:T ratio of 50:1 was compared by Student's t-test.

FIG. 4. Immunisation with rHBsAg DNA encoding a RSV CTL epitope at the ΔGLS site evokes RSV-directed effector and memory CTL responses.

(A) Epitope-specific IFN-γ-secreting T-cell response following immunisation with recombinant HBsAg plasmid DNA encoding a RSV CTL epitope. H-2^{d} mice (BALB/c, 3 per group) were immunised twice subcutaneously (sc) with rHBsAg DNA encoding the M2 15-mer (pM2₁₅ΔIPQ) RSV CTL epitope at the site of deleted GLS HBsAg CTL epitope, as indicated. Control H-2^{b} were immunised once im with GLS peptide plus adjuvant. IFN-γ-secreting cells were quantified by ELISPOT assay using splenocytes harvested at 14 days (DNA immunisations) or 10 days (peptide immunisations) after immunisation, and incubated for 15-18 h with or with-out specific peptide (M2₁₅ or GLS) as shown. (B) Epitope specific CTL responses following immunisation with recombinant HBsAg plasmid DNA encoding a RSV CTL epitope. H-2^{d} mice and H-2^{b} mice (3 per group) were immunised as above. Percent cytotoxicity of splenocytes restimulated with M2₁₅ or GLS peptide was measured in a ⁵¹Cr release assay using P815 or EL4.A2 target cells respectively pulsed with M2₁₅ or GLS peptide, respectively, or without peptide as shown. Data points represent means+/−standard deviations of three replicates. (Note in some cases standard deviation bars, though plotted are too small to appear). For ELISPOT, IFN-γ secretion with and without peptide was compared by Student's t-test. For CTL assay, cytotoxicity against targets with and without peptide at an E:T ratio of 50:1 was compared by Student's t-test.

FIG. 5. Immunisation with rHBsAg DNA encoding a RSV CTL epitope evokes protection in mice challenged with RSV. H-2^{d} mice (BALB/c, 5 per group) mice were immunised twice id with pM2₁₅ΔIPQ DNA or with pM2₁₅ΔIPQ DNA. Control groups were immunised twice sc with pHBsAg wild-type DNA, or once sc with M2₁₅ peptide+adjuvant. Immunised mice were challenged 2 weeks (for DNA immunised mice) or 10 days (for peptide immunised mice) later with 8×10⁵ pfu RSV intranasally. Four days later mice were euthanased and lungs removed (A) Representative lung histology of RSV infected mice (i); unimmunised mouse, (ii); pM2₁₅ΔGLS DNA immunised mouse, (iii); pM2₁₅ΔIPQ DNA immunised mouse, (iv) normal mouse lung without RSV challenge. Lungs were fixed, paraffin sectioned and stained with hematoxylin-eosin. (B) RSV-specific immunostaining of lung cyrosections stained in indirect immunofluorescence by sequential incubations with goat anti-RSV antibody and FITC-anti goat Ig. (C) There was reduced viral load and histopathology in vaccinated mice indicating that the rHS-RSV vaccine is protective.

FIG. 6. Immunisation with rHBsAg DNA encoding a CTL epitope of the E7 oncoprotein of human papillomavirus epitope evokes E7-directed effector and memory CTL responses, and protects against challenge with an E7-expressing tumour.

(A) Epitope-specific IFN-γ-secreting T-cell response following immunisation with recombinant HBsAg plasmid DNA encoding HPV 16 E7 CTL epitope. H-2^{b} mice (A2.K^{b}, 3 per group) were immunised twice sc with pRAHΔIPQ DNA encoding the RAH epitope CTL epitope of HPV 16. Control groups H-2^{b} were immunised twice with pHBsAg wild-type DNA or one im with GLS peptide plus adjuvant. IFN-γ-secreting cells were quantified by ELISPOT assay using splenocytes harvested at 14 days (DNA immunisations) or 10 days (peptide immunisations) after immunisation, and incubated for 15-18 h with or without specific peptide (RAH) as shown. Histogram bars represent means+/−standard deviation of 3 replicates. (B) Epitope specific CTL responses following immunisation with rHBsAg plasmid pRAHΔIPQ DNA Mice (3 per group) were immunised as above. Percent cytotoxicity of splenocytes restimulated with RAH peptide was measured in a ⁵¹Cr release assay using EL4.A2 target cells pulsed with RAH peptide or without peptide as shown. Data points represent means+/−standard deviations of three replicates. (Note standard deviation bars, though plotted, are too small to appear). For ELISPOT, IFN-γ secretion with and without peptide was compared by Student's t-test. For CTL assay, cytotoxicity against targets with and without peptide at an E:T ratio of 50:1 was compared by Student's t-test. (C) Growth of E7-expressing tumour in mice immunised with pRAHΔIPQ DNA expressing the RAH E7 CTL epitope, and controls. Mice immunised with pRAHΔIPQ DNA, with pHBsAg wild-type DNA, with RAH peptide+adjuvant, or unimmunised, were challenged with 2×10⁵ E7-expressing TC-1 tumour cells on day 14 post-immunisation. Results are expressed as tumour-free mice (%) at the indicated time points. Differences between the Kaplan Meier survival curves were calculated using the log-rank statistic.

FIG. 7. Immunisation with rHBsAg DNA encoding two copies of an inserted foreign epitope does not elicit enhanced CTL responses to the inserted epitope compared with rHBsAg encoding a single copy.

(A) H-2^{d} mice (BALB/c, 3 per group) were immunised three times id with rHBsAg DNAs encoding the M2₁₅ CTL epitope of RSV F protein at either one location (pM2₁₅ΔIPQ.HWI or pM2₁₅ΔGLS) or at two locations (pM2₁₅ΔIPQΔGLS.HWI) (Table 1). Control mice were immunised with pHBsAg wild-type DNA (pHBsAg.W/T). IFN-7-secreting cells were quantified by IFN-γ ELISPOT assay using splenocytes harvested four weeks after third immunisation, and incubated for 15-18 h with or without M2₁₅ peptide. (B) M2-specific CTL memory responses following immunisation with rHBsAg plasmid DNA encoding RSV M2 CTL epitope. Groups of mice (3 per group) were immunised as above. Percent cytotoxicity of splenocytes restimulated for six days with M2₁₅ peptide was measured in a ⁵¹Cr release assay using P815 target cells pulsed with M2₁₅ peptide or without peptide as shown. Data points represent means of 3 replicates+/−standard deviation.

FIG. 8. Immunisation with rHBsAg DNA encoding two protective CTL epitopes from distinct pathogens elicits CTL responses to both epitopes.

(A) Epitope-specific IFN-γ-secreting T-cell response of ex vivo splenocytes following two subcutaneous immunisations of (H-2^{d}×H-2^{b}) F1 mice (BALB/c×A2.1K^{b}, 3 per group) with rHBsAg DNA encoding both M2₁₅ and RAH epitopes (pM2₁₅ΔIPQ,RAHΔGLS.HWI), or encoding either the M2₁₅ epitope alone (pM2₁₅ΔIPQ) or the RAH epitope alone (pRAHΔGLS). IFN-γ-secreting cells were quantified by ELISPOT assay using splenocytes incubated for 15-18 h with or without specific peptide M2₁₅ and/or RAH as shown. Histogram bars represent means+/−standard deviation of 3 replicates. (B) Percent cytotoxicity of splenocytes restimulated with RAH peptide or with M2₁₅ peptide were measured in a ⁵¹Cr release assay using P815 or EL4.A2 target cells pulsed with M2₁₅ peptide or RAH peptide respectively, or without peptide, as shown. Data points represent means+/−standard deviations of three replicates. (Note standard deviation bars, though plotted, are too small to appear in some instances). (C) Growth of E7-expressing tumour in (H−2d×H−2b) F1 mice immunised with pM2₁₅ΔIPQ.RAHΔGLS.HWI DNA, with pRAHΔIPQ DNA, or with control DNA as indicated. Mice were challenged with 8×10⁵ E7-expressing TC-1 tumour cells on day 14 post-immunisation. Results are expressed as tumour-free mice (%) at the indicated time points. Differences between the Kaplan Meier survival curves were calculated using the log-rank statistic.

FIG. 9. Antibody response to rHBsAg DNA immunogen encoding a B-mimotope is down-regulated when the rHBsAg DNA simultaneously encodes inserted CTL epitopes. H-2^{d} mice (BALB/C, 8 per group) were immunised three times at two weekly intervals with 100 ug (id.) rHBsAg plasmid DNAs pHWI, pM2₁₅ΔIPQΔGLS.HWI which encode the RSV F protein B-cell mimotope HWSISKPQ (Table 1) in the HBsAg surface ‘a’-loop, or with pHBsAg wild-type DNA. Four weeks after the final immunisation, sera were sampled and antibody titre evaluated in ELISA assay using HWI mimotope MAP-coated plates. Results are presented for the sera of individual mice.

FIG. 10. Immunization with chimeric HBsAg DNA encoding either one or more copies of a single tumour-associated CTL epitope. A. Epitope-specific IFN-γ secreting T cell responses following immunization as quantified by ELISPOT analysis restimulated splenocytes. B. Epitope-specific CTL responses as measured by chromium release assay

FIG. 11. A. Epitope-specific IFN-γ secreting T cell responses following immunization with chimeric HBsAg DNA encoding RSV and hMPV CTL epitopes as quantified by ELISPOT analysis of splenocytes ex vivo.B. Epitope-specific IFN-γ secreting T cell responses following immunization with chimeric HBsAg DNA encoding an RSV and hMPV CTL epitopes or with RSV or hMPV peptide as quantified by ELISPOT analysis of splenocytes post in vitro restimulation.

FIG. 12 A. Epitope-specific IFN-γ secreting T cell responses following immunization with peptides to M2, SH and N as quantified by ELISPOT analysis of splenocytes ex vivo. B. Epitope-specific IFN-γ secreting T cell responses following immunization with RSV or hMPV peptide as quantified by ELISPOT analysis of splenocytes post in vitro restimulation.

FIG. 13 Epitope-specific Cytotoxic T cell responses in mice immunized with a chimeric HBsAg DNA vaccine encoding RSV and hMPV CTL epitopes or with RSV or hMPV peptide

FIG. 14 Epitope-specific Cytotoxic T cell responses in mice immunized with WT-HBsAg (control) with a chimeric HBsAg DNA vaccine encoding a tumour-associated antigen (pHPVT(SIL) at the SIL site, or with E7 peptide.

FIG. 15 A. Epitope-specific IFN-γ secreting T cell responses following immunization with chimeric HBsAg DNA encoding a tumour-associated antigen (RAH) at the SIL site, with E7 peptide, or unvaccinated, as quantified by ELISPOT analysis of splenocytes ex vivo. B. Epitope-specific IFN-γ secreting T cell responses following immunization with chimeric HBsAg DNA encoding a tumour-associated antigen (RAH) at the SIL site, with E7 peptide, or unvaccinated as quantified by ELISPOT analysis of splenocytes post in vitro restimulation.

FIG. 16 % tumour free mice following immunization with chimeric HBsAg DNA encoding a tumour-associated antigen (RAH) at the SIL site, with RAH peptide, or unvaccinated, and subsequently challenged with TC-1 tumour cells.

FIG. 17 Therapeutic control of TC-1 tumour in tumour-bearing mice subsequently vaccinated with chimeric HBsAg DNA encoding a tumour-associated antigen (RAH) at the SIL site, with RAH peptide, or unvaccinated.

FIG. 18 A, B Ex vivo γ-IFN Elispot, chromium release cytotoxicity and tumour protection in mice immunized with (A) chimeric HBsAg DNA encoding a tumour-associated antigen (RAH) at the IPQ site, and (B) with RAH peptide.C. Ex vivo γ-IFN Elispot, chromium release cytotoxicity and tumour protection in mice immunized with chimeric HBsAg DNA encoding a tumour-associated antigen (RAH) at the GLS site.

FIG. 19. Nucleotide and amino acid sequences of wild-type HBsAg and HBsAg chimeric constructs with single or multiple copies of RAH foreign epitopes. In addition to the inserted foreign epitopes, spacer sequences are included in some cases (see for example ARAHYNIVTFAL, AARAHYNIVTFAL and AARAHYNIVTFA) and also manipulation of restriction sites may also introduce amino acid sequence changes, or combinations of these (see for example the ANSGAA in HBsAg.RAHx3 and HBsAg.RAHx5). Sequence of the substituted HBsAg construct may include in some cases sequence of foreign T cell epitopes (for example in HBsAg.RAHx1 and HBsAg.RAHx3) and B cell epitopes (such as in HBsAg.RAHx1, HBsAg.RAHx3 and HBsAg.RAHx5) without determination of their effect on T cell responses. Epitope sequences are underlined.

FIG. 20. Nucleotide and amino acid sequences of wild-type HBsAg and HBsAg chimeric constructs having CTL epitopes from different pathogens RSV, HPV, EBV, HIV and CMV. In addition to the inserted foreign epitopes, spacer sequences are included in some cases as previously described. Epitope sequences are underlined.

FIG. 21. Nucleotide and amino acid sequences of wild-type HBsAg and HBsAg chimeric constructs having CTL epitopes from pathogens RSV and metapneumovirus (mouse and human). Epitope sequences are underlined.

FIG. 22. Nucleotide and amino acid sequences of wild-type HBsAg and HBsAg chimeric constructs having CTL epitopes substituted by site-directed mutagenesis. Epitope sequences are underlined.

### DETAILED DESCRIPTION OF THE PREFERRED EMBODIMENT

Previous studies that derived HBsAg VLP immunogens containing foreign CTL epitopes have relied upon insertion of the foreign sequences at sites where endogenous restriction endonuclease (RE) sites are located in the HBsAg gene or attachment of the foreign sequences to the amino or carboxy terminals of the HBsAg gene. These insertions led to unpredictable stability and/or secretability of the recombinant particles, probably due to conformational changes in the recombinant HBsAg molecule.

The inventors have discovered that substitution of endogenous T cell epitopes of HBsAg with foreign T cell epitopes with similar physical properties (i.e. size and hydrophobicity) results in less structural change of the HBsAg molecule and thereby helps to conserve HBsAg conformation and subsequent stability/secretability. Substitution of HBsAg CTL epitopes with foreign CTL epitopes may ensure efficient processing of the inserted foreign sequence. Also, after optimisation, several epitopes can be efficiently delivered within one HBsAg protein, and several different proteins may form one VLP. This is difficult to achieve with HBsAg proteins with N-terminal or C-terminal extensions that represent domains of foreign proteins.

By “substitution” is meant that at least part or all of a HBsAg epitope (i.e an endogenous epitope) is removed, deleted or excised and replaced with an epitope of a protein other than HBsAg (i.e an exogenous or foreign epitope and/or that an endogenous epitope of HBsAg is replaced with multiple copies of said endogenous epitope and/or one or more copies of a different HBsAg epitope. Only the epitope or part of the epitope per se is selectively substituted.

For example, referring to the prior art, when a foreign epitope is inserted at a restriction site most or all of the HBsAg epitope sequence remains intact even though the epitope sequence subsequently may be non-contiguous. According to the present invention, when an epitope is substituted at least part or all of the epitope is removed and replaced with a foreign epitope.

The inventors have developed a novel, generic delivery system using HBsAg which is applicable to immunotherapy of infectious diseases and cancers which induce a T cell response (preferably a CTL response) and, optionally, an antibody response in a host. Immunogenic T cell epitopes derived from one or more proteins other than HBsAg are incorporated into precise regions of HBsAg.

A specific endogenous T cell epitope of HBsAg is deleted or excised and substituted with a foreign T cell epitope, i.e. the endogenous T cell epitope is replaced by a foreign T cell epitope. In addition, the endogenous T cell epitope is replaced with a foreign T cell epitope of similar structural characteristics, for example size and phobicity, i.e. replacing “like with like”. This permits retention of tertiary conformation and retention of structural integrity within the HBsAg molecule.

The present invention further contemplates co-delivery of foreign T and B cell epitopes by means of substitution of endogenous B cell epitopes in HBsAg with foreign B cell epitopes.

This strategy also exploits the powerful inherent immunogenicity of HBsAg which facilitates generation of immunogenic responses in an animal.

The present inventors have demonstrated that immunisation with recombinant HBsAg DNA afforded protection against RSV infection or HPV 16 E7 tumours in mice. The inventors also demonstrated that immunisation with DNA encoding HBsAg comprising both RSV- and HPV E7-CTL epitopes elicited CTL responses to both foreign epitopes and afforded protection against both RSV infection and HPV E7 expressing tumour in mice. The recombinant HBsAg DNA immunogens elicited effector and memory CTL responses in vitro and pathogen- and tumour-protective responses in vivo, when the recombinant HBsAg DNAs were used to immunise mice.

The inventors further demonstrated that recombinant HBsAg DNA for both RSV- and HPV-CTL epitopes elicited simultaneous immune responses to both epitopes in mice. These data demonstrate the efficacy of HBsAg DNA as a vaccine delivery system to deliver disease relevant protective CTL responses.

The results also demonstrate that HBsAg can be used to deliver a plurality of T cell epitopes derived from one or a plurality of pathogenic origins. The results have generic implications for the further development of HBsAg as a multivalent vaccine vector. As proof of principle, the inventors have demonstrated that antigen-specific T cell responses are generated to CTL epitopes from RSV and hMPV delivered using this system.

There are also specific implications for vaccines against respiratory syncytial virus (RSV) infection and human papillomavirus (HPV)-associated carcinoma.

### Isolated Proteins

As hereinbefore described the invention provides an isolated protein whereby one or more HBsAg T cell epitopes may be substituted by one or more T cell epitopes from any disease causing pathogen.

The isolated protein of the invention may be referred to hereinafter as a “substituted HBsAg chimera”.

It will be appreciated that the invention contemplates HBsAg derived from a variety of viruses belonging to the family Hepadnaviridae such as but not limited to members of the genera Avidepadnavirus or Orthohepadnavirus. Non-limiting examples of family members include hepatitis B virus, duck hepatitis B virus, woodchuck hepatitis B virus, ground squirrel hepatitis B virus, woolley monkey hepatitis B virus and snow goose hepatitis B virus.

By “protein” is meant an amino acid polymer. Amino acids may include natural (i.e genetically encoded), non-natural, D- and L-amino acids as are well known in the art.

A “peptide” is a protein having less than fifty (50) amino acids.

A “polypeptide” is a protein having (50) or more amino acids.

Specific HBsAg epitopes, which are optimal for epitope substitution may be selected for substitution. The HBsAg epitopes include, but are not limited to, IPQ, GLS, FLL, VLQ, FLG, LLD and SIL. Other epitopes are listed in the following references: Loirat et al. 2000. J. Immunology 165: 4748, Sette et al. 2001. J. Immunology 166: 1389; Roh et al. 2001. Virus Res. 73: 17-26; Schirmbeck et al. 2001, J. Immunology, 166, 1405-1413.

Prima facie, the value of each individual epitope for substitution HBsAg is not readily apparent. Surprisingly, the inventors have demonstrated that, for example, insertion at the SIL site is only useful when performed in isolation. If substitution of the SIL site is performed in parallel with other sites, the immunogenic potential of the SIL site is lost. Moreover, the immunogenic efficacy of each site is not equal as a T cell epitope substituted at a particular site may be immunodominant over a T cell epitope inserted at another site. In a particular embodiment, a CTL epitope inserted at IPQ is immunodominant over a CTL epitope inserted at GLS.

A particular feature of the invention is that substitution of specific HBsAg epitopes appear to provide VLPs with increased stability and secretion competence. The inventors have investigated four regions of the HBsAg molecule; GLS, IPQ, FLL (Table 1) and SIL. Substitution of the GLS epitope appears to provide VLPs with increased stability and secretion competence.

The invention also contemplates substitution with corresponding epitopes of hepatitis B virus strains having variant sequences. Examples of hepatitis B variant epitopes can be found in the following references and databases: National Library of Medicine (NCBI); Ali Fares and Holmes 2002, Journal of Molecular Evolution 54, 807-814; Galibert et al. 1979, Nature 281, 646-650, HBV subtype ayw genome: Accession number: V01460, J02203; Okamoto et al., 1986, Journal of General Virology 67: 2305-2314, HBV subtype ayr genome: Accession number: NC_{—}003977; Ono et al. 1983, Nucleic Acids Res. 11: 1747-1757, Accession number: AJ748098, Accession number: D00630, Accession number: V00866.

The invention therefore contemplates variant isolated proteins and encoding nucleic acids sharing an appropriate level of sequence identity with isolated proteins and encoding nucleic acids set forth herein.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

In one embodiment, a variant of the substituted HBsAg chimera comprises at least 80% sequence identity, preferably at least 85% or 90% sequence identity and more preferably at least 95%, 96%, 97%, 98% or 99% sequence identity. It will be appreciated that a variant comprises all integer values less than 100%, for example the percent value as set forth above and others. Variants of the substituted HBsAg chimera include, for example, HBsAg from subtypes as set forth above or HBsAg derived other members of the genus orthohepadnavirus or members of the avihepadnavirus genus.

The invention also contemplates use of a fragment of the substituted HBsAg chimera or encoding nucleic acid.

In one embodiment, a “fragment” includes a protein comprising an amino acid sequence that constitutes less than 100% of an amino acid sequence of an entire protein. A fragment preferably comprises less than 99%, 98%, 97%, 96%, 95%, 94%, 93%, 92%, 91%, 90%, 85%, 80% or 75%, but greater than 70%, of the entire protein.

Preferably, the fragment is a “biologically active” fragment, which retains biological activity of a given protein, or an encoding nucleic acid. More preferably, the biologically active fragment has the ability to self-assemble and form VLPs.

It will be appreciated that one or more HBsAg T cell epitopes can be substituted with one or more T cell epitopes from any disease causing pathogen. For example, one, two, three, four, five or six endogenous T cell epitopes may be substituted. Preferably, one, two or three epitopes are substituted. More preferably, one or two epitopes are substituted.

Epitopes may be derived from, be obtainable from or otherwise be of any pathogen or tumour for which disease-specific or disease-associated epitopes are or will be known. It appreciated that a plurality of foreign epitopes may be utilised. Preferably, one, two, three, four, five or six foreign epitopes are substituted into HBsAg. More preferably, one or two foreign epitopes are substituted into HBsAg.

Examples of references that describe potentially suitable epitopes are as follows: RSV: Hsu, et al. 1999, J. Gen. Virol., 80, 1401-1405; Rotavirus: Choi, et al. 2002, Vaccine 20, 3310-3321; Mycobacterium: Tascon, et al. 1996, Nat. Med. 2, 888-892; Measles: Jaye, et al. 2003, J. Virol. 77 5014-5016; Malaria: Arevalo-Herrera & Herrera, 2001, Mol. Immunol. 38, 443-455; Human papillomavirus: Kast, et al. 1994, J. Immunol. 152, 3904-3912; Hepatitis C virus: Ward, et al. 2002, Clin. Exp. Immunol. 128, 195-203 and Table 4; Influenza virus: Deliyannis et al., 2002, JV, 76, 4212-4221; Human metapneumovirus: Herd, et al. 2006 J. Virol. 80, 2034-2044 and CMV: WO 03/000720.

The present invention further provides a vehicle for induction of a humoral immune response by inclusion of one or more foreign B cell epitopes in HBsAg. As exogenous B cell epitopes are recognized by the B cell receptor, the epitope must be exposed on the surface of the antigen. The HBsAg molecule contains two major hydrophilic and, presumably, exposed domains. One of these domains contains a highly immunogenic region, referred to as the ‘a’-determinant, which carries the major B cell epitopes of the protein. The a-determinant is located in a double-loop structure. It can be readily appreciated by a person of skill in the art that an array of recombinant DNA technology methods can be used to insert foreign B cell epitopes into the aforementioned domains. The use of restriction enzyme sites provides a convenient and efficient system to introduce heterologous sequence into HBsAg nucleic acid. It can be contemplated that naturally-occurring restriction enzyme sites may be utilized using, as an example, the method of Delpeyroux et al (J Mol Biol, 1987, 195: 343-350), which is incorporated herein by reference. Alternatively, one or more foreign restriction enzyme sites can be engineered into a desired position within either domain, thereby providing a more precise insertion point, which may aid with structural conformation limitations. US Application No. 2003/0211996 provides a useful example of such a strategy and is incorporated herein by reference.

Suitable epitopes include either well characterized conformational or linear epitopes or alternatively, a mimotope. Plotnicky-Gilquin et al (J Virol, 1999, 73: 5637) and Power et al (Vaccine, 2001, 19: 2345) each provide non-limiting examples of suitable antigen-specific B cell epitopes and are incorporated herein by reference. Preferably the B cell epitope is a mimotope with a sequence consisting of HWSISKPQ.

The substituted HBsAg chimera may also comprise one or more additional amino acid sequences. “Additions” of amino acids may include fusion of a HBsAg protein of the invention or a fragment thereof with other proteins or peptides. The other protein may, by way of example, assist in the purification of the protein. For instance, these include a polyhistidine tag, maltose binding protein (MBP), green fluorescent protein (GFP), Protein A or glutathione S-transferase (GST). Other additions include “epitope tags” such as FLAG and c-myc epitope tags.

Well known examples of fusion partners include, but are not limited to, glutathione-S-transferase (GST), Fc portion of human IgG, maltose binding protein (MBP) and hexahistidine (HIS₆), which are particularly useful for isolation of the fusion protein by affinity chromatography. For the purposes of fusion protein purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system.

In some cases, the fusion partners also have protease cleavage sites, such as for Factor Xₐ or Thrombin, which allow the relevant protease to partially digest the fusion protein of the invention and thereby liberate the recombinant protein of the invention therefrom. The liberated protein can then be isolated from the fusion partner by subsequent chromatographic separation.

In another embodiment, the substituted HBsAg chimera may include spacer sequence for example ARAHYNIVTFAL, AARAHYNIVTFAL and AARAHYNIVTFA) and also manipulation of restriction sites may also introduce amino acid sequence changes, or combinations of these.

The recombinant HBsAg protein may be conveniently prepared by a person skilled in the art using standard protocols as for example described in Sambrook et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5 and 6.

The invention also contemplates chemical derivatives of the substituted HBsAg chimera, such as produced using techniques described in CURRENT PROTOCOLS IN PROTEIN SCIENCE Chapter 15, for example.

### Isolated Nucleic Acids and Expression Constructs

It will be appreciated from the foregoing and also from pharmaceutical compositions described in more detail hereinafter, that the invention also provides use of an isolated nucleic acid encoding the HBsAg protein of the invention. Non-limiting examples of isolated nucleic acids are provided in FIGS. 19 to 22.

The term “nucleic acid” as used herein designates single- or double-stranded mRNA, RNA, cRNA, RNAi and DNA inclusive of cDNA and genomic DNA and DNA-RNA hybrids. Nucleic acids may also be conjugated with fluorochromes, enzymes and peptides as are well known in the art.

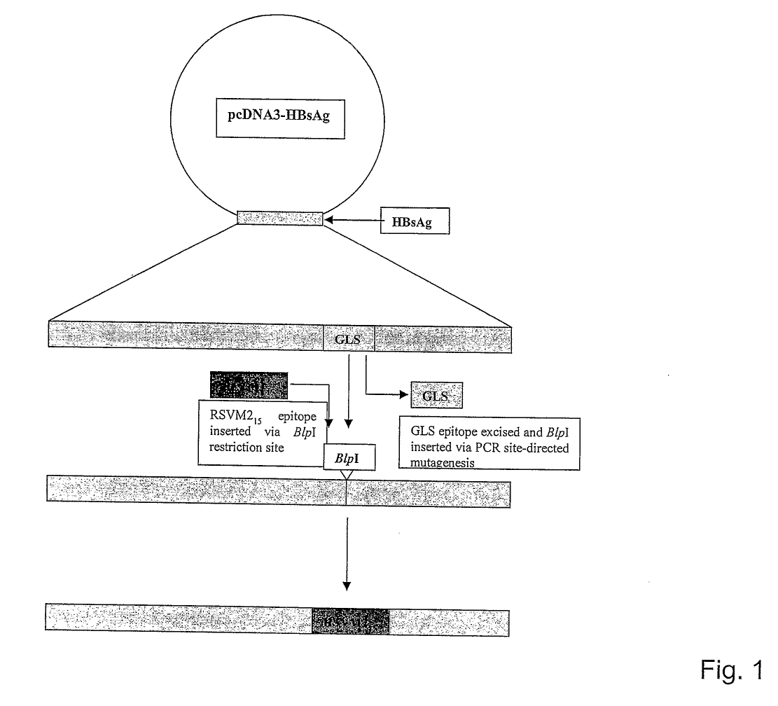

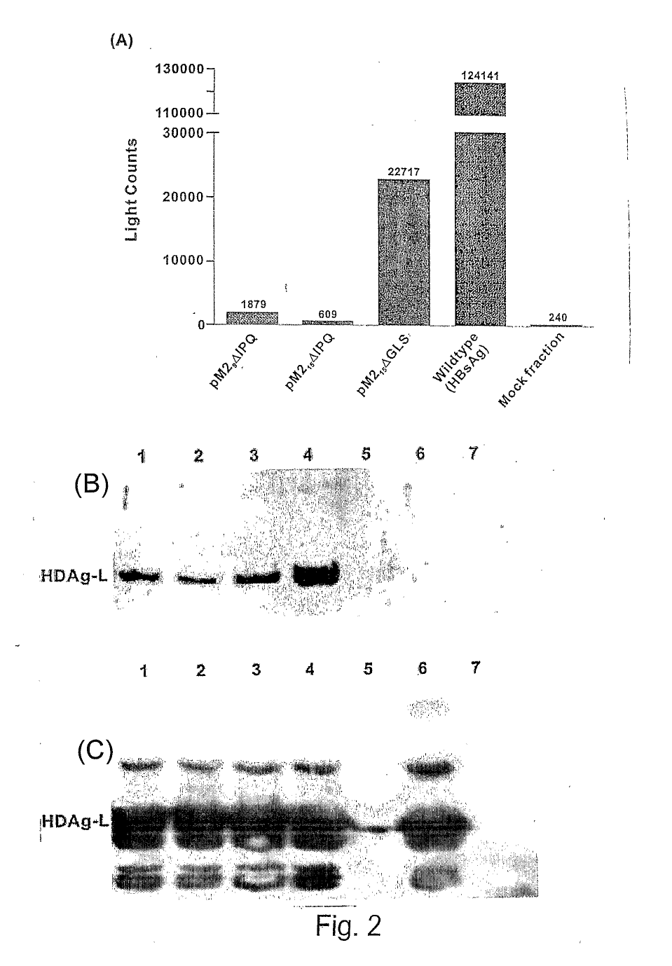

It will be appreciated that a nucleic acid encoding the substituted HBsAg chimera of the invention may readily be produced using methods known in the art. An example of the preparation of such nucleic acids is shown in FIG. 1 and the Materials and Methods section of the Examples.

The invention also contemplates variant HBsAg nucleic acids having one or more codon sequences altered by talking advantage of codon sequence redundancy.

A particular example of a variant HBsAg nucleic acid is optimization of a nucleic acid sequence according to codon usage, as is well known in the art. This can effectively “tailor” a nucleic acid for optimal expression in a particular organism, or cells thereof, where preferential codon usage has been established.

In certain embodiments, said isolated HBsAg nucleic acid may be present in an expression construct, wherein said isolated nucleic acid is operably linked or connected to one or more regulatory sequences in an expression vector. Furthermore, expression from said expression construct may be performed in a prokaryotic or eukaryotic system.

In one particular embodiment, the expression construct is suitable for expression of the substituted HBsAg chimera in bacteria such as E. coli.

In another particular embodiment, the expression construct is for expression in a system of eukaryotic origin such as one or more mammalian cells, tissues or organs in vitro or in vivo, inclusive of cells capable of producing VLPs. Ideally, although not exclusively, the expression construct is suitable for use in a hepatocyte-derived cell line. Non-limiting examples include HepG2, Hep3B and HuH-7 cell lines. Preferably, the cell capable of producing VLPs is HuH-7.

In yet another particular embodiment, the expression construct is able to drive expression of substituted HBsAg chimera in a yeast cell. Examples of suitable yeast expression strains include Pichia pastoris, Saccharomyces cerevisae and Kluyveromyces lactis but are not limited thereto.

In still another embodiment, the invention contemplates introduction of said expression construct into plant cells in order to produce transgenic plants able to deliver a plant-derived oral vaccine. Streatfield S J Immunol. Cell Biol. 2005, 83, 257-262 provides an excellent review of the field and is incorporated herein by reference.

In a further embodiment, the expression construct is for administration of the HBsAg nucleic acid to an individual.

An “expression vector” may be either a self-replicating extra-chromosomal vector such as a plasmid, or a vector that integrates into a host genome, inclusive of vectors of viral origin such as adenovirus, lentivirus, poxvirus and flavivirus vectors as are well known in the art.

By “operably linked or connected” is meant that said regulatory nucleotide sequence(s) is/are positioned relative to the recombinant nucleic acid of the invention to initiate, control, regulate or otherwise direct transcription and/or other processes associated with expression of said nucleic acid.

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, splice donor/acceptor sequences, enhancer or activator sequences and nucleic acid packaging signals.

Constitutive promoters (such as CMV, SV40 and human elongation factor promoters) and inducible/repressible promoters (such as tet-repressible promoters and IPTG-, alcohol-, metallothionine- or ecdysone-inducible promoters) are well known in the art and are contemplated by the invention, as are tissue-specific promoters such as α-crystallin promoters. It will also be appreciated that promoters may be hybrid promoters that combine elements of more than one promoter (such as SRα promoter).

The expression construct may also include a fusion partner (typically provided by the expression vector) so that the substituted HBsAg chimera is expressed as a fusion protein with said fusion partner, as hereinbefore described.

Expression constructs may also include a selection marker nucleic acid that confers transformed host cell resistance to a selection agent. Selection markers useful for the purposes of selection of transformed bacteria include bla, kanR and tetR while transformed eukaryotic cells may be selected by markers such as hygromycin, G418 and puromycin, although without limitation thereto.

Expression constructs may be introduced into cells or tissues, inclusive of cells capable of VLP production, by any of a number of well known methods typically referred to as “transfection” “transduction”, “transformation” and the like. Non-limiting examples of such methods include transformation by heat shock, electroporation, DEAE-Dextran transfection, microinjection, liposome-mediated transfection (e.g. lipofectamine, lipofectin), calcium phosphate precipitated transfection, viral transformation, protoplast fusion, microparticle bombardment and the like.

### Pharmaceutical and Immunotherapeutic Compositions, Vaccines and Methods of Treatment

The invention provides a substituted HBsAg chimera that exploits the powerful inherent immunogenicity of HBsAg and facilitates the generation of immunogenic responses in an animal. Therefore the invention provides use of the substituted HBsAg chimera for the treatment of all pathogenic diseases and cancers, which induce a protective T or B cell response in an animal. Such infectious diseases include those mediated by HIV, RSV, rotavirus, cytomegalovirus, Epstein Barr virus, HPV, measles virus, malaria, hepatitis C virus, hepatitis B virus, human metapneumovirus, Mycobacterium tuberculosis, parainfluenza virus, influenza virus, plasmodium falciparum. Furthermore, various cancers such as melanoma and hepatocellular carcinoma can be treated by the invention.

A particular aspect of the invention relates to use of the substituted HBsAg chimera as a vaccine delivery system. However, the invention more broadly provides a pharmaceutical composition not limited to use in vaccine delivery, but inclusive of immunotherapeutic compositions in several forms:

(i) VLPs comprising RNA produced by the expression construct of the invention;

(ii) purified or substantially pure isolated protein of the invention (substituted HBsAg chimera); or

(iii) a plasmid DNA expression construct of the invention directing transcription of RNA in vivo.

The pharmaceutical composition may further comprise a pharmaceutically-acceptable carrier, diluent or excipient.

Such compositions may be delivered for the purposes of generating immunity, preferably protective immunity, to pathogens such as viruses, bacteria and parasites although without limitation thereto.

In an alternative embodiment, immunotherapeutic treatment of cancers, such as cervical cancer caused by HPV, is contemplated by the present invention.

In a preferred form, the pharmaceutical composition of the invention is an immunogenic composition comprising an isolated protein, isolated nucleic acid or VLP and an immunologically acceptable carrier, diluent or excipient.

By “immunogenic” is meant capable of eliciting an immune response, preferably a protective immune response, upon administration to a host.

Thus in a particular form, the immunogenic composition of the invention may be a vaccine which induces a protective immune response when administered to a host.

The present invention is readily amenable to production of a mixed population of virus-like particles by means of introduction of one or more expression constructs into a cell. For example, the invention contemplates introduction of at least two separate expression constructs, one of which encodes HBsAg substituted with one or more T cell epitopes and the other construct encodes HBsAg comprising one or more B cell epitopes. A particular advantage conferred by this system is the possibility to substitute HBsAg with a plurality of foreign epitopes, thereby producing multivalent vaccine delivery system. In a particular embodiment, the present invention provides for a VLP-based protective vaccine for paediatric respiratory diseases in which RSV and hMPV are major pathogens.

In yet further embodiments, VLPs described may be as follows: (1) a VLP may comprise all of the same HBsAg construct (this could be same T-epitope with/without B-epitope, (2) a VLP may comprise a mixture of different HBsAg constructs assembling into a heterogeneous VLP comprising the different forms of the HBsAg protein, or (3) a mixture of VLPs wherein either (1) or (2) are included; this would include a heterogeneous mixture of different VLPs comprising homogeneous HBsAg. VLPs as hereinbefore described may be useful in pharmaceutical compositions, vaccines and methods of treatment.

It will be appreciated that compositions, vaccines and methods of treatment may be used therapeutically or prophylactically.

Suitably, the immunogenic composition and/or vaccine of the invention is administrable to an animal host, inclusive of domestic animals, livestock, performance animals and humans.

Any suitable procedure is contemplated for producing vaccine compositions. Exemplary procedures include, for example, those described in New Generation Vaccines (1997, Levine et al., Marcel Dekker, Inc. New York, Basel, Hong Kong) which is incorporated herein by reference.

As hereinbefore described, the immunogenic composition and/or vaccine of the invention may include an “immunologically-acceptable carrier, diluent or excipient”.

Useful carriers are well known in the art and include for example: thyroglobulin; albumins such as human serum albumin; toxins, toxoids or any mutant crossreactive material (CRM) of the toxin from tetanus, diphtheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, a T cell epitope of a bacterial toxin, toxoid or CRM may be used. In this regard, reference may be made to U.S. Pat. No. 5,785,973 which is incorporated herein by reference.

The “immunologically-acceptable carrier, diluent or excipient” includes within its scope water, bicarbonate buffer, phosphate buffered saline or saline and/or an adjuvant as is well known in the art. Suitable adjuvants include, but are not limited to: surface active substances such as hexadecylamine, octadecylamine, octadecyl amino acid esters, lysolecithin, dimethyldioctadecylammonium bromide, N,N-dicoctadecyl-N′, N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminum phosphate, aluminum hydroxide or alum; lymphokines, QuilA and immune stimulating complexes (ISCOMS).

Any safe route of administration may be employed for providing a patient with the immunotherapeutic composition of the invention. For example, oral, rectal, parenteral, sublingual, buccal, intravenous, intra-articular, intramuscular, intra-dermal, subcutaneous, inhalational, intraocular, intraperitoneal, intracerebroventricular, transdermal and the like may be employed. Intra-muscular and subcutaneous injection is appropriate, for example, for administration of immunogenic compositions and vaccines.

The above compositions may be administered in a manner compatible with the dosage formulation, and in such amount as is immunologically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

Immunotherapeutic compositions of the invention may be used to prophylactically or therapeutically immunize animals such as humans.

However, other animals are contemplated, preferably vertebrate animals including domestic animals such as livestock and companion animals.

As will be described in more detail hereinafter, vaccines of the present invention may be in the form of VLPs or DNA.

Immune responses may be induced against viruses, tumours, bacteria, protozoa and other invertebrate parasites by expressing appropriately immunogenic proteins and peptide epitopes inclusive of polyepitopes using the vaccine of the invention.

Preferably, the immune response involves induction of antibodies, CD8+ CTLs and/or CD4+ T cells.

More preferably, the immune response involves induction of long term effector CD8+ CTLs.

In one particular embodiment, the present inventors have demonstrated that immunization with a DNA-based VLP vaccine encoding the ESYIGSINNITKQSA CTL epitope from RSV protected mice against RSV-associated pulmonary disease following RSV intranasal challenge.

In another particular embodiment, the present inventors have demonstrated that immunization with DNA-based VLP vaccine encoding the RAHYNIVTF CTL epitope from HPV16 E7 protected mice against growth of a challenge inoculation of HPV16 E7 expressing tumour.

It will also be appreciated that immunotherapeutic compositions and vaccines of the invention may, in certain embodiments, include an adjuvant.

As will be understood in the art, an “adjuvant” means one or more substances that enhances the immunogenicity and/or efficacy of a vaccine composition. Non-limiting examples of suitable adjuvants include squalane and squalene (or other oils of animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran or with aluminium phosphate; carboxypolymethylene such as Carbopol' EMA; acrylic copolymer emulsions such as Neocryl A640 (e.g. U.S. Pat. No. 5,047,238); vaccinia or animal poxvirus proteins; sub-viral particle adjuvants such as cholera toxin, or mixtures thereof.

So that the invention may be readily understood and put into practical effect, the skilled person is directed the following non-limiting examples.

### EXAMPLES

### Example 1

### Materials and Methods

### Cloning Procedures

DNA encoding HBsAg-S was cloned into pcDNA3 (Invitrogen) to derive plasmid pcD3-HBsAgS as described (Netter et al., 2001 J. Virol. 75: 2130-2141). Two intermediate vectors, generically termed pcD3-HBsAgS-Δ‘X’, where Δ‘X’ represents deleted DNA encoding either the IPQ epitope or the GLS epitope of HBsAg (Table 1), were derived from pcD3-HBsAgS by PCR-driven site-directed mutagenesis. The oligonucleotide primers were designed to insert a unique restriction enzyme (RE) site at the site of deletion of the epitope-encoding DNA (NheI at ΔIPQ and BlpI at ΔGLS, respectively) (FIG. 1). A post-PCR DpnI RE digest was carried out to eliminate the original DNA template in the PCR mixture. Plasmid pM2₁₅ΔIPQ was derived by insertion of DNA encoding 15 amino acids representing the H-2^{d}-restricted RSV M2 extended epitope (ESYIGSINNITKQSA) at the NheI site of pcD3-HBsAgS-ΔIPQ. The DNA insert was derived by annealing of two corresponding oligonucleotides followed by ligation into the dephosphorylated pcD3-HBsAgS-ΔIPQ vector linearised by NheI digestion. Plasmids pM2₉ΔIPQ and pRAHΔIPQ (Table 2), were derived by a similar procedure, inserting the M2₉ CTL epitope of RSV and the RAH CTL epitope of HPV at the ΔIPQ site, respectively. Plasmid pM2₁₅ΔGLS was derived by insertion of DNA encoding M2₁₅ at the BlpI site of pcD3-HBsAgS-ΔGLS. Plasmid pM2₁₅ ΔIPQΔGLS was derived by sequential insertions of DNA encoding the M2₁₅ epitope at ΔIPQ and ΔGLS sites. Plasmid pM2₁₅ΔIPQ.RAHΔGLS was derived by sequential insertions of DNA encoding the M2₁₅ epitope and the RAH epitope at ΔIPQ and ΔGLS sites respectively. Plasmid pM2₁₅ΔIPQ RAHΔGLS.HWI was derived by inserting a RSV B mimotope (Table 1) into the AgeI site in the hydrophilic ‘a’-loop of pcD3-HBsAgS as described (Netter et al., 2001 J. Virol. 75, 2130-2141), followed by sequential insertions of DNA encoding the M2₁₅ epitope and the RAH epitope at ΔIPQ and ΔGLS sites respectively. Plasmids were grown in Top 10 bacterial cells (Invitrogen) under ampicillin selection according to standard procedures. Plasmid DNA was purified as Wizard Plus SV Minipreps (Promega) or Endofree Plasmid Megepreps (Qiagen). All plasmids were verified by RE analysis and DNA sequencing (PCR Sprint Temperature Cyclin System-Hybaid).

### Transfection

HuH-7 cells were transfected with bacterial plasmid DNA using calcium phosphate. The supernatant was harvested 5 days later, and HBV surface antigen (HBsAg) was measured by the Abbott Prism HBsAg assay (Abbott Diagnostics). The level of HBsAg in the cell culture fluid was quantified by comparison with a commercially available vaccine (Engerix-B; 20 ug/ml; Smith-Kline Beecham). The transfection efficiency of different plasmids was normalised to the activity of secreted alkaline phosphatase (SEAP) as previously described (Berger, et al. 1988, Gene, 66, 1-10). The variation in the range of SEAP was less than two-fold. The presence of hepatitis delta large antigen (HDAg-L) was identified by immunoblot using a human anti-delta virus antibody, and assayed by the ECL-plus detection system (Amersham). Mice

A2.IKb mice express a chimeric HLA class 1 molecule, A2.1Kb on a predominantly C57B1 (H-2b) background. A2.1K^{b} mice are capable of making CTL responses restricted through both HLA A*0201 and H-2^{b} class 1 molecules. BALB/c mice make CTL responses restricted through H-2^{d}. Mice were housed under specific pathogen free conditions. (A2.IKb×BALB/c) F1 hybrid mice make CTL responses restricted through H-2^{b} and H-2^{d}. Mice were used at 7-15 weeks of age, but within a given experiment were littermates or closely age and sex matched.

### Peptides

Peptides were synthesised with free ends using 9-fluorenylmethoxycarbonyl (F-moc) chemistry and analysed by HPLC and by amino acid analysis. Peptide stocks were made at 10 mg/ml in dimethyl sulphoxide and diluted into tissue culture medium for assays. The RSV F protein mimotope was synthesised as a tetrameric MAP construct as described (Chargelegue, et al. 1998, J. Virol., 72, 2040-2046). All syntheses were carried out by Chiron Corporation (Melbourne, Australia).

### Immunisation of Mice and Restimulation Of Splenocytes for CTL

For DNA immunisations, anaesthetized (ketamine/xylazine) mice were injected twice at three weekly intervals intradermally (id.) in the ear with 10 ug of purified plasmid DNA. Three weeks later, spleens were removed and splenocytes were restimulated in vitro for 6 days in 24 well tissue culture plates (5×10⁶ cells per well) in the presence of 1 ug/ml cognate peptide. For peptide immunisations, mice were immunised subcutaneously (sc.) at the tail base with 50 ug peptide+0.25 ug tetanus toxoid (TT) as a source of T-helper epitopes+10 ug Quil A adjuvant. Ten days later spleens were harvested and splenocytes were restimulated as above.

### Cells

EL4.A2 cells were derived by transfection of EL4 cells with A2.1Kb plasmid encoding the chimeric MHC class I heavy chain (above), as described previously (Doan, et al. 1998, Virology, 244, 352-364). EL4.A2 cells are susceptible to specific CTL lysis through both H-2^{b} and HLA A*0201 restriction pathways. P815 (mastocytoma cell line) is susceptible to specific CTL lysis through H-2d restriction pathway. HuH-7 is a human hepatoma cell line. Cells were maintained in RPMI medium (Gibco) supplemented with 2 mM glutamine, 1 mM sodium pyruvate, 20 mM HEPES, 5×10⁻⁵ M β-mercaptoethanol, 100 IU/ml penicillin, 100 ug/ml. streptomycin and 10% foetal bovine serum.

### CTL Assays.

CTL assays were conducted as previously described (Doan et al., 1998, Virology 244: 352-364). In summary, target cells (10⁴ per well) sensitised at 37° C. for 1 hour with 1 ug/ml cognate or irrelevant peptide, or medium alone, and labelled with 100 uCi ⁵¹Chromium (Cr), were incubated with effector cells at various effector: target cell ratios in triplicate in 96 well microtitre plates. Negative controls included wells containing target cells but no effector cells (=‘background’). Supernatants were harvested from CTL assays at 4 hours, and ⁵¹Cr release quantified by gamma counting. Results are expressed as percent cytotoxicity+/−standard deviation (⁵¹Cr release in experimental wells minus background/detergent-mediated total release minus background)×100%. Experimental and control values were compared for significant difference using Student's t-test.

### ELISPOT.

Epitope-specific gamma interferon (IFN-γ) secreting spleen cells were enumerated ex vivo by an enzyme-linked immunospot (ELISPOT) assay with minimal CD8+ T-cell epitope peptides. Microwell plates (MultiScreen-HA, Millipore, North Ryde, NSW, Australia) were coated with capture antibody (rat anti-mouse IFN-γ, clone RA-6A2, BD PharMingen, San Diego, Calif.), blocked with DMEM 10% FBS, and washed. Serial dilutions of spleen cells, starting at 10⁶/well were added, with 100 U/mL recombinant human interleukin-2 (Sigma, Castle Hill, NSW, Australia) and incubated with 10 μg/mL peptide or without peptide, for 18 h at 37° C. The cells were then lysed, the plates washed, and detection antibody (biotinylated anti-mouse IFN-γ, clone XMG 1.2, BD PharMingen) was added for 2 h. at 37° C. After further washing, plates were developed using streptavidin-alkaline phosphatase (BD PharMingen) and BCIP-NBT substrate (Sigma FAST™ 5-bromo-4-chloro-3-indolylphosphate-nitroblue tetrazolium). IFN-γ spots were counted using an AID EliSpot reader system. Results were calculated as IFN-γ positive cells/10⁶ spleen cells. Experimental and control values were compared for significant difference using analysis of variance (ANOVA).

Evaluation of RSV infection.

Lightly anaesthetised mice were inoculated intranasally (in.) with 8×10⁵ plaque forming units (pfu) RSV/mouse in 20 ul PBS. Four days later mice were euthanased and lungs removed and divided into three portions. The first portion was formalin fixed, sectioned and histochemically stained with haematoxylin-eosin. The second portion was cryosectioned and subject to indirect immunofluorescence using goat anti-RSV antibody and FITC-anti goat Ig detecting antibody (Chemicon). The third portion was snap-frozen in ethanol/dry-ice, stored at −70 degrees, pending determination of RSV titre by immunofocus assay.

### Immunofocus Assay

Serial dilutions (10⁰ to 10⁻⁵) of supernatants from homogenised lung were added to monolayers of HEp-2 cells in serum-free DMEM medium in microtitre trays. After 2 hrs incubation to allow virus adsorption, 50 ul of DMEM with 2% FCS was added and further incubated at 37° C. in 5% CO₂ incubator for 40 hours. Following washing, cells were fixed in 80% acetone and air-dried. Wells were blocked with PBS+5% skim milk powder, prior to sequential addition of goat anti-RSV (Chemicon; 1:200 dilution) and anti-goat Ig horse radish peroxidase (HRP)-conjugated (Chemicon), with appropriate incubations (37 degrees, 30 mins) and intervening washing steps. The reaction was developed with DAB (tetra-aminobiphenyl hydrochloride) substrate (Sigma) and Urea/H₂O₂/NaCl according to manufacturer's instructions (Sigma). The reaction was stopped by washing the wells with dH₂O. Plaques were counted and viral concentration calculated using the following formula: Average number of plaques per well×dilution factor×20=pfu/ml of stock.

### Tumour Protection Assays.

To assay tumour prevention, groups of H-2^{b} mice (5 per group) were immunised with 10 ug plasmid DNA id, or 100 ug of E7 peptide+tetanus toxoid+Quil A subcutaneously (sc.). TC-1 cells (2×10⁵ in 0.1 mL Hank's buffered salt solution) were subsequently injected sc. on the left flank using a 21 gauge needle. (The tumour dose was pre-determined by titration experiments to discern a minimal dose giving rise to tumour in 100% of unimmunised mice). Tumour growth was monitored every 2 days, and mice were euthanased when tumour volume exceeded 1000 mm³. Unimmunised mice received the same number of cells and served as a control. Data are represented as Kaplan Meier curves of % tumour free mice at given time points after tumour injection.

### ELISA

ELISA plates (Nunc: Maxisorp 96-wells) were coated with HBsAg-S particles (10 ng/50 ul) or with RSV B mimotope peptide (10 ug/ml) in PBS overnight at 4° C. Following incubation with blocking buffer and sequential incubations with serial dilutions of mouse serum followed by anti-mouse IgG conjugated to HRP (Sigma Chemicals) with intervening washes, the plates were developed with ABTS (2,2′-Azino-bis(3-ethylbenz-thiazoline-6-sulfonic acid) substrate solution. Colour change was read at 410 nm using an automatic plate reader (Murex). A result was considered positive where the experimental optical density (OD) exceeded the OD of the negative control (pre-bleed serum) by a factor of two plus two standard deviations.

### Results

### Epitope Replacement Allows for Efficient Formation and Secretion of VLPS.

In a first set of experiments, the inventors replaced either the IPS or the GLS epitopes of HBsAg-S (Table 1), with the M2 CTL epitope of RSV matrix protein. Both were chosen as a ‘strong’ epitopes in the native HBsAg particle (Schirmbeck et al., 2002, J. Immunol. 168, 6253-6262; Loirat et al., 2000, J. Immunol. 165: 4748-4755. The minimal M2 CTL epitope of RSV is a 9 mer represented by M2 residues 82-90 (designated M2₉); a larger 15 mer peptide sequence represented by residues 81-95 (designated M2₁₅) also functions as a CTL epitope (Table 1 and Hsu et al., 1995, Immunology 85, 347-350; Hsu Steward Immunology 1995, 85). To insert M2₉ or M2₁₅ into HBsAg, nucleotide sequence encoding the endogenous IPQ or GLS CTL epitope was deleted and a unique RE site was artificially inserted at the site of deletion to derive an intermediate vector DNA, as described in the Materials and Methods. Nucleotides encoding M2₉ or M2₁₅ were cloned into the intermediate vector at these sites to produce pM2₉ΔIPQ, pM2₁₅ΔIPQ and pM2₁₅ΔGLS, respectively (Table 2).

The inventors examined whether pM2₉ΔIPQ, pM2₁₅ΔIPQ and pM2₁₅ΔGLS (Table 2) were able to encode secretable rHBsAg VLPs. Individual plasmid DNAs were transfected into HuH-7 cells which were cultured for 5 days. Culture fluid was then harvested and tested for secreted HBsAg by an ice vitro chemiluminescent immunoassay based on an anti-HBsAg IgM antibody (Abbott Prism). A representative result with corrected light counts is shown in FIG. 1. The replacement of the endogenous IPQ epitope by RSV M2₉ or M2₁₅ RSV epitope resulted in a major reduction in secretion compared with the wild type HBsAg. Insertion of RSV M2₁₅ at the GLS deleted site resulted in a lesser reduction in secretion though still ca 6-fold lower than that of wild type HBsAg.

The reduction in light counts may have represented either decreased secretion efficiency of the recombinant HBsAg-RSV proteins or reduced affinity of the anti-HBsAg IgM antibody as a result of the replacement RSV epitopes perturbing overall conformational structure of the rHBsAg. To address this we conducted co-transfections of HuH-7 cells with a plasmid expressing the hepatitis delta virus large antigen (HDAg-L). Hepatitis delta virus (HDV) is an RNA agent which exists as a sub-viral satellite of hHBV. The envelope of HDV is provided by HBV. Co-expression of HDAg-L and HBsAg-S in tissue culture cells results in incorporation (packaging) of HDAg-L into the empty HBsAg-S particles (OMalley & Lazinski, 2002, J. Virol. 76:10060-10063). Only in the packaged form is HDAg-L secreted (Wang et al., 1991, J. Virol. 65, 6630-6636).

In the co-transfection experiments, secretion was determined by HDAg-L immunoblot detection in the supernatant (FIG. 2B). Non-secreted HDAg-L in the cell pellet was also measured (FIG. 2C). HDAg-L was confirmed in the supernatant of cells which had been co-transfected with rHBsAg, (FIG. 2B, lanes 1-3), although less than in cells co-transfected with wild-type HBsAg FIG. 1B, lane 4). No HDAg-L was detected in the supernatant of cells transfected only with the plasmid encoding HDAg-L, although HDAg-L was clearly present in the cell pellet, confirming that in the absence of HBsAg, HDAg-L was not secreted (FIG. 2 lane 6). Neither was HDAg-L present in the supernatant of cells transfected with the plasmid encoding HBsAg alone (FIG. 2B, lane 5). This result indicates that rHBsAg in which the endogenous IPQ or GLS CTL epitopes are replaced by a foreign CTL epitope (RSV M2) retain the structural features necessary for HDAg-L/HBsAg interaction, measured by the functional capacity to package HDAg-L for secretion. Together, the data in FIG. 2 demonstrate that replacing an endogenous HBsAg CTL epitope with a foreign CTL epitope at either of two sites allows the assembly and secretion of structurally sound recombinant HBsAg particles, although the efficiency of secretion was apparently less than that of wild-type HBsAg.

Insertion into the HBsAg Gene of the DNA Encoding RSV M2 CTL Epitope at the ΔIPQ Site Elicits Effector and Memory M2-Directed CTL Responses when the rHBsAg DNA is Used to Immunise Mice

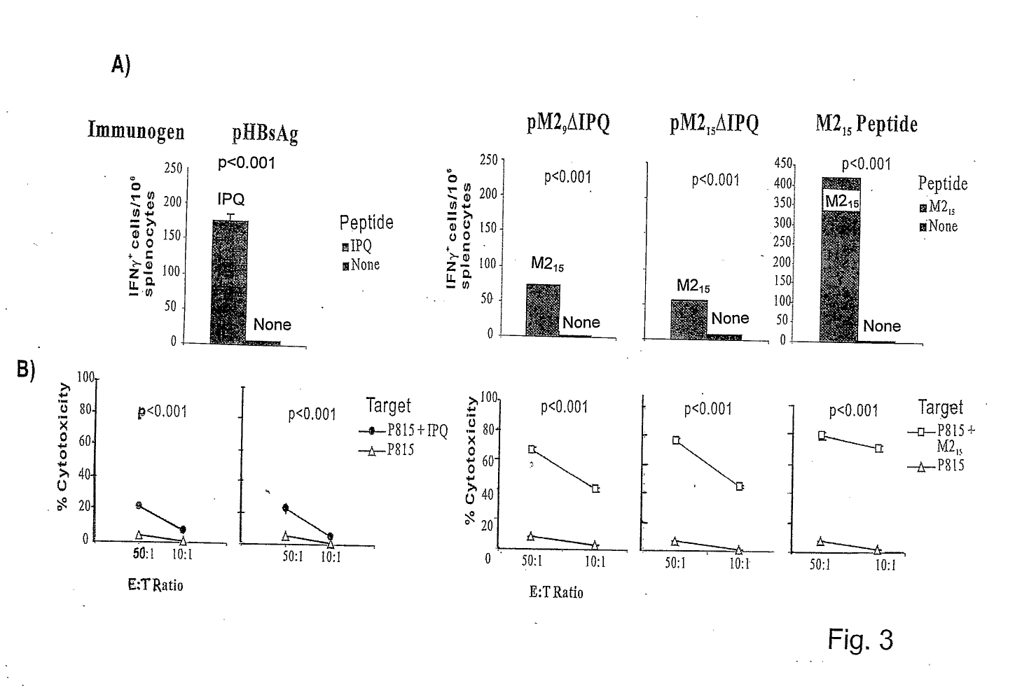

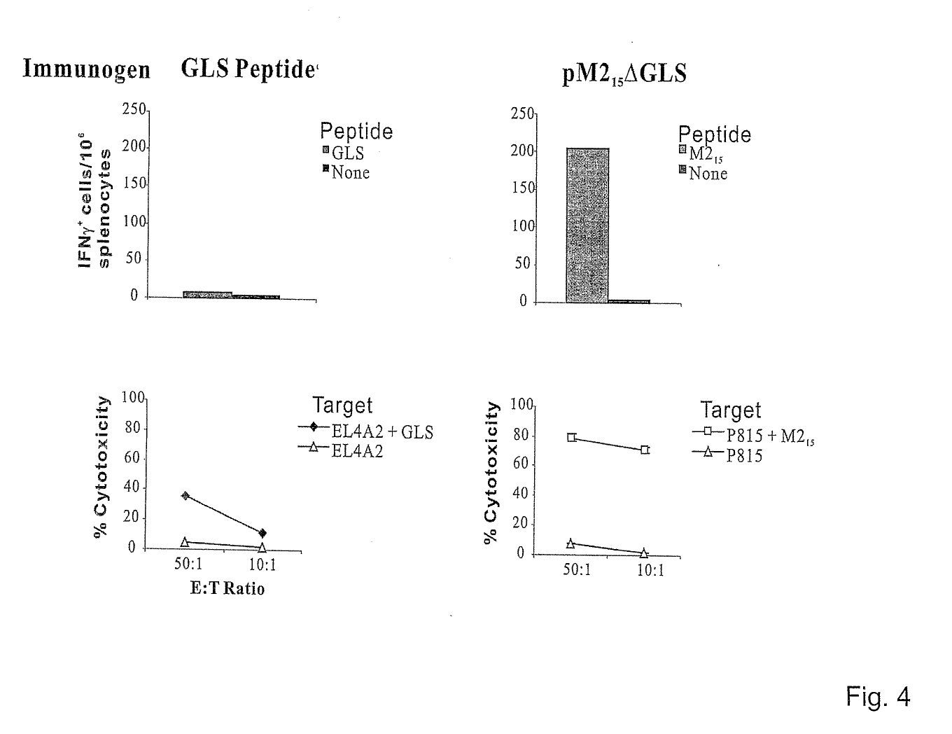

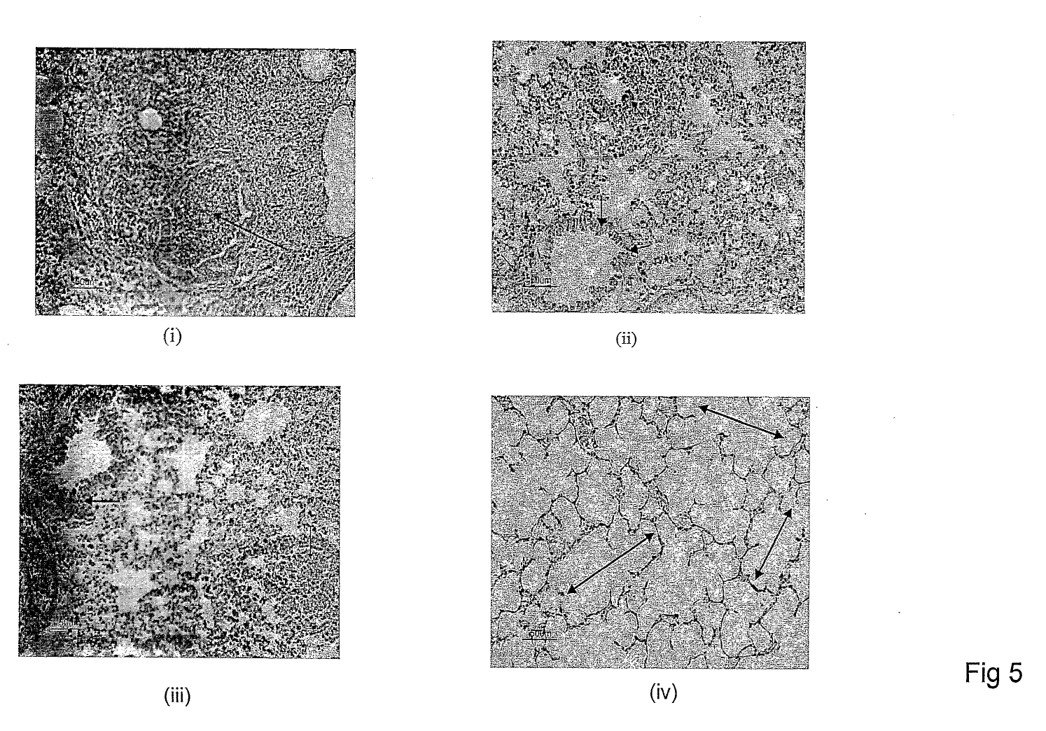

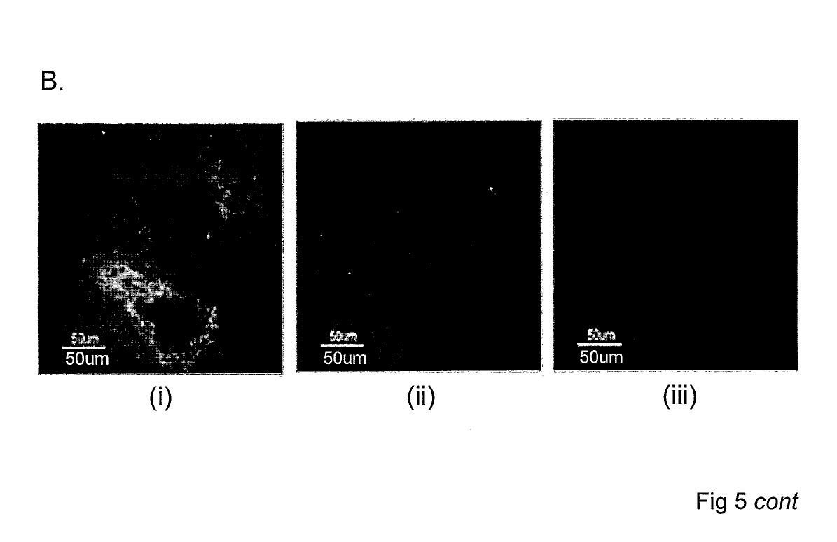

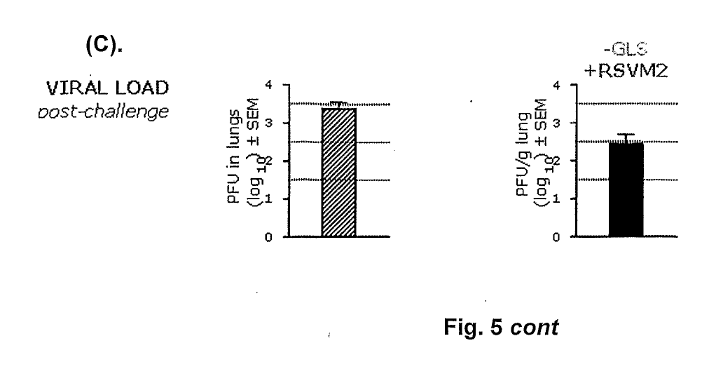

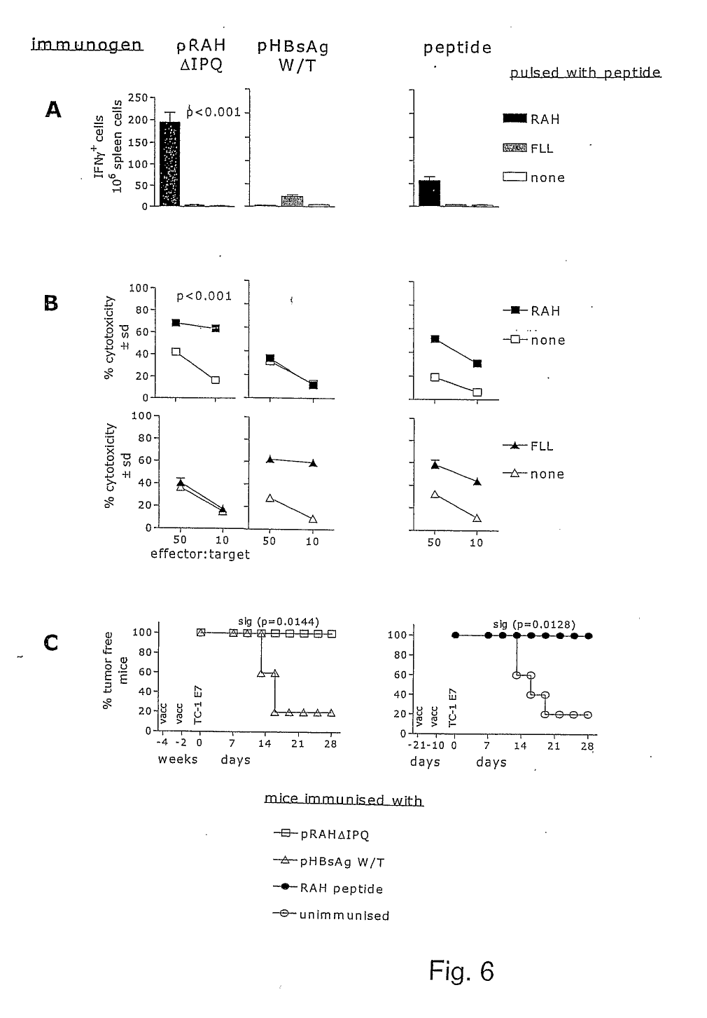

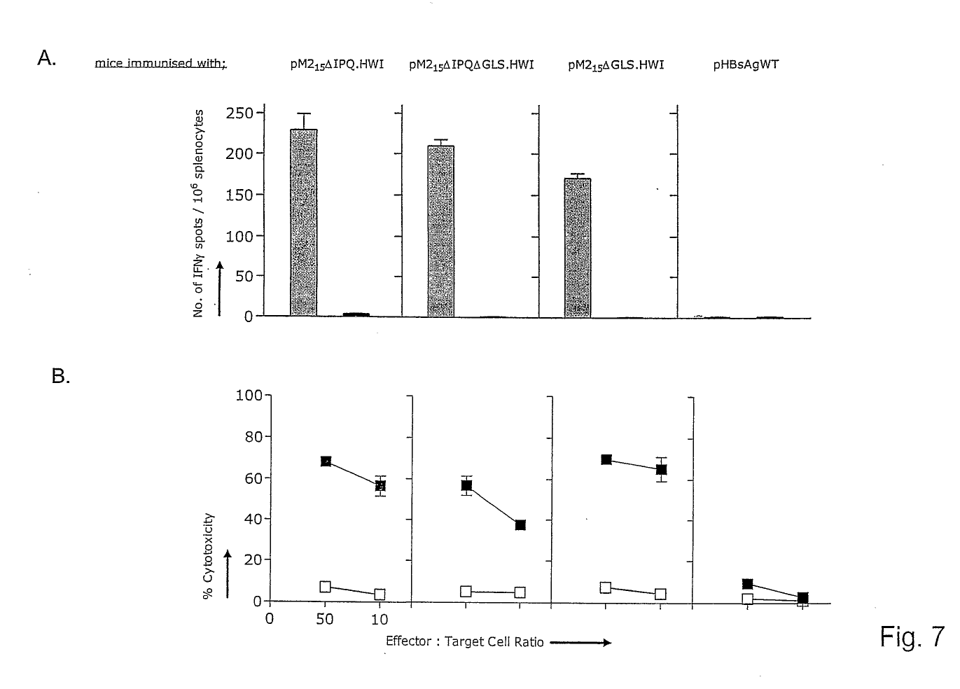

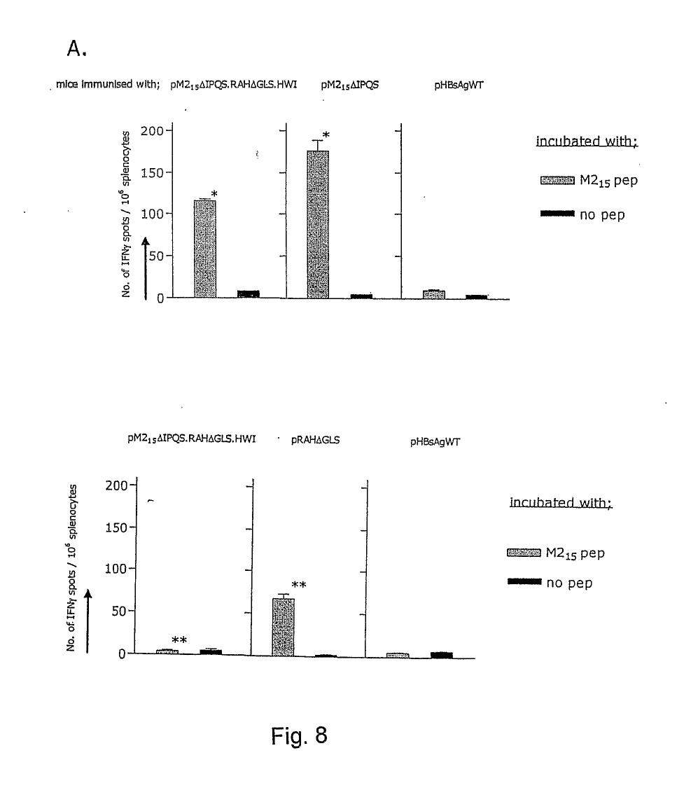

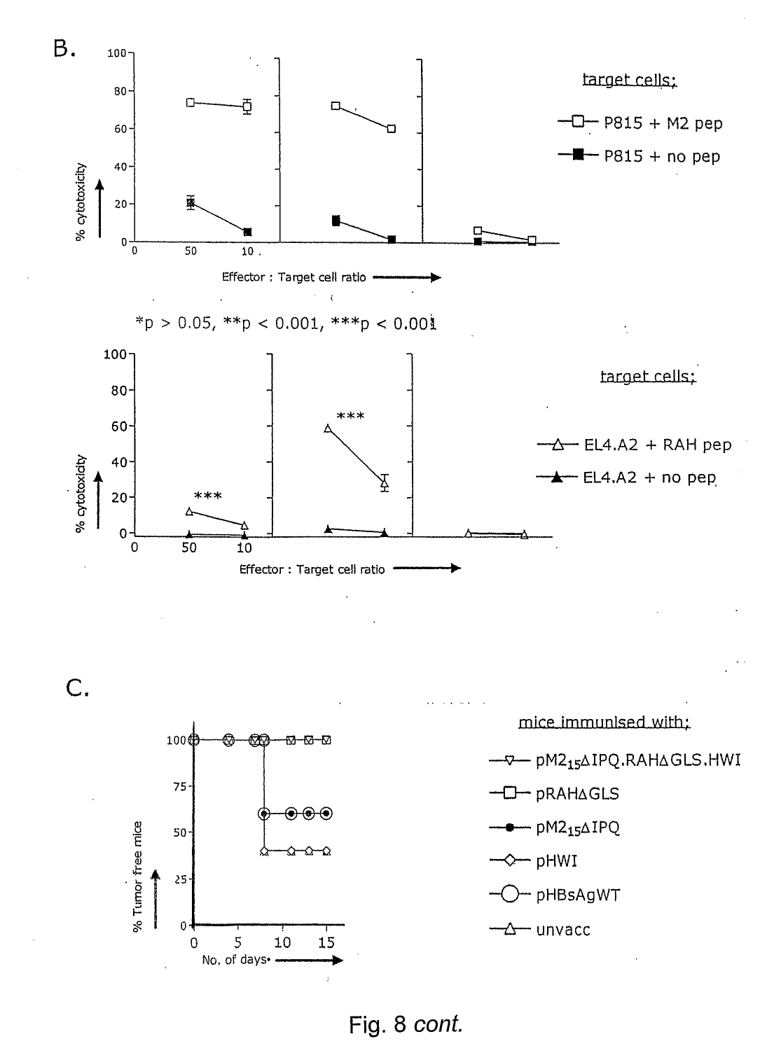

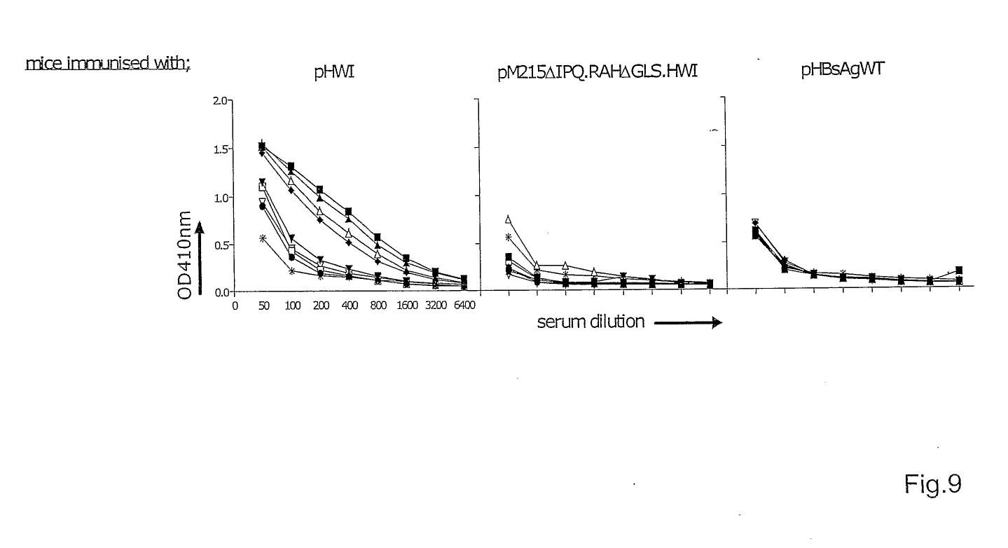

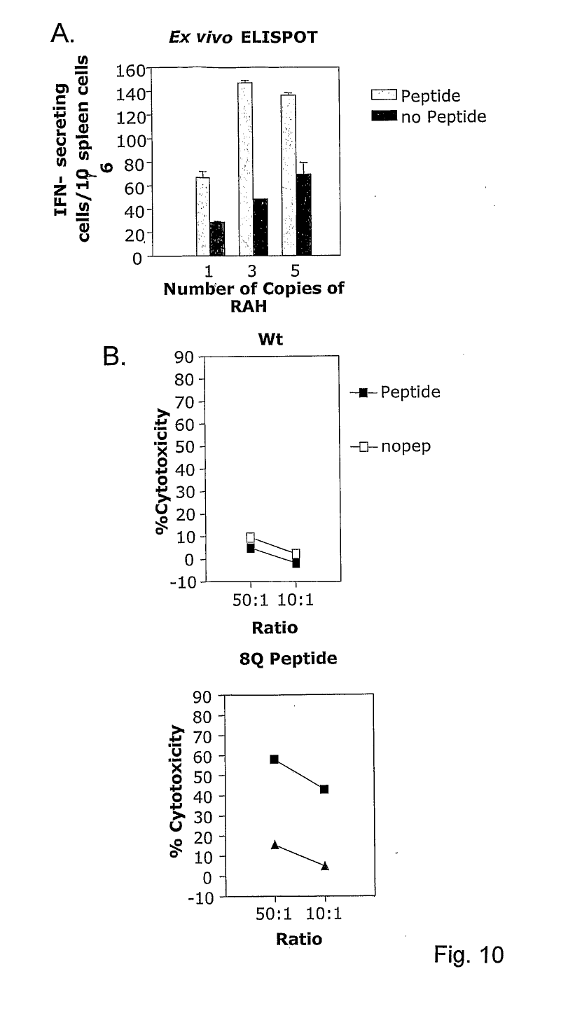

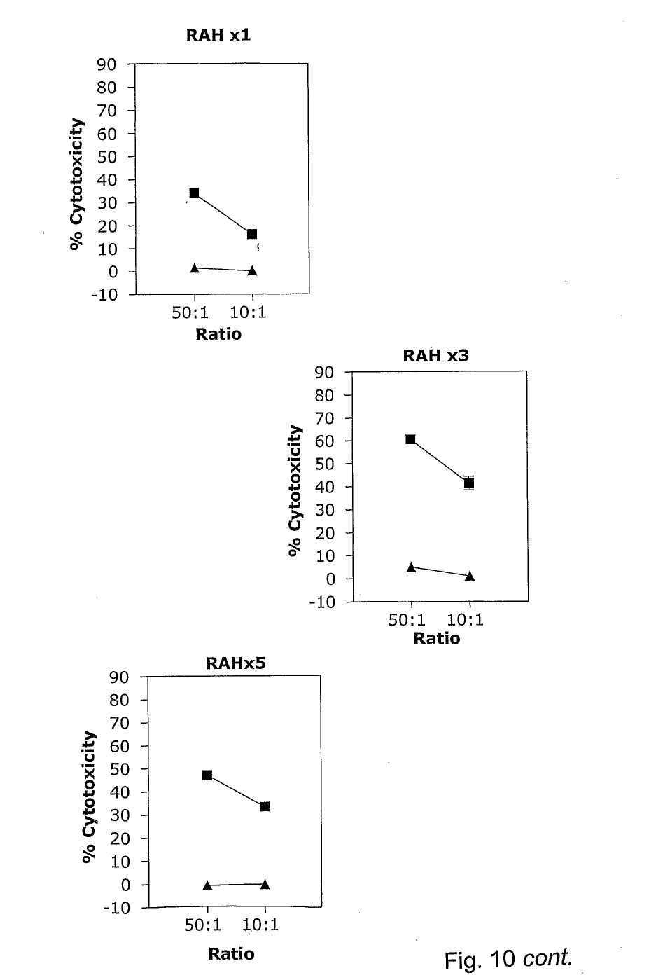

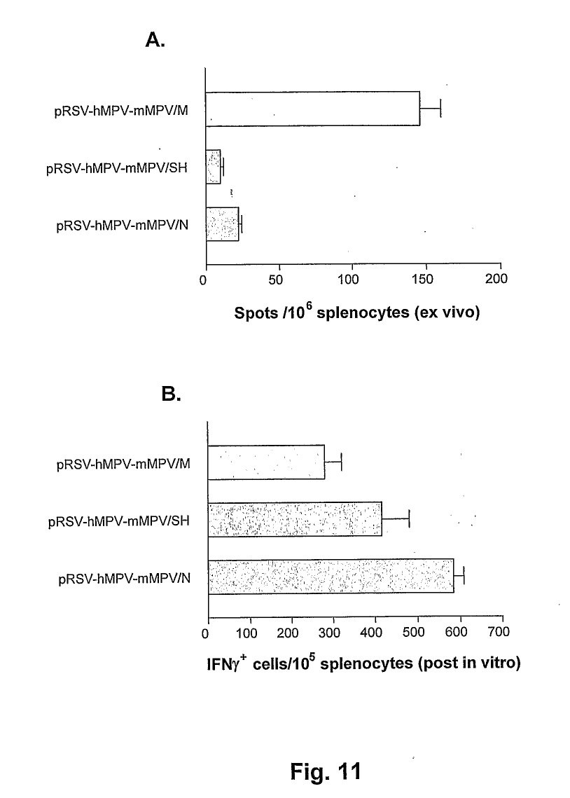

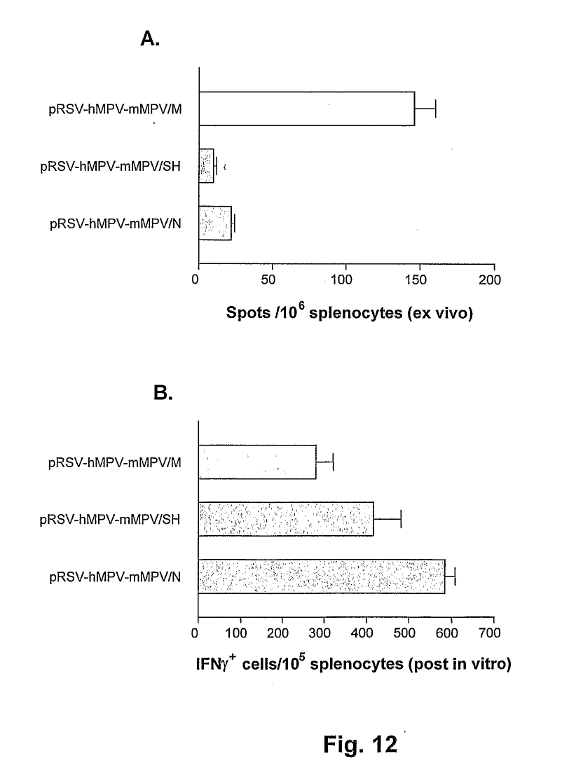

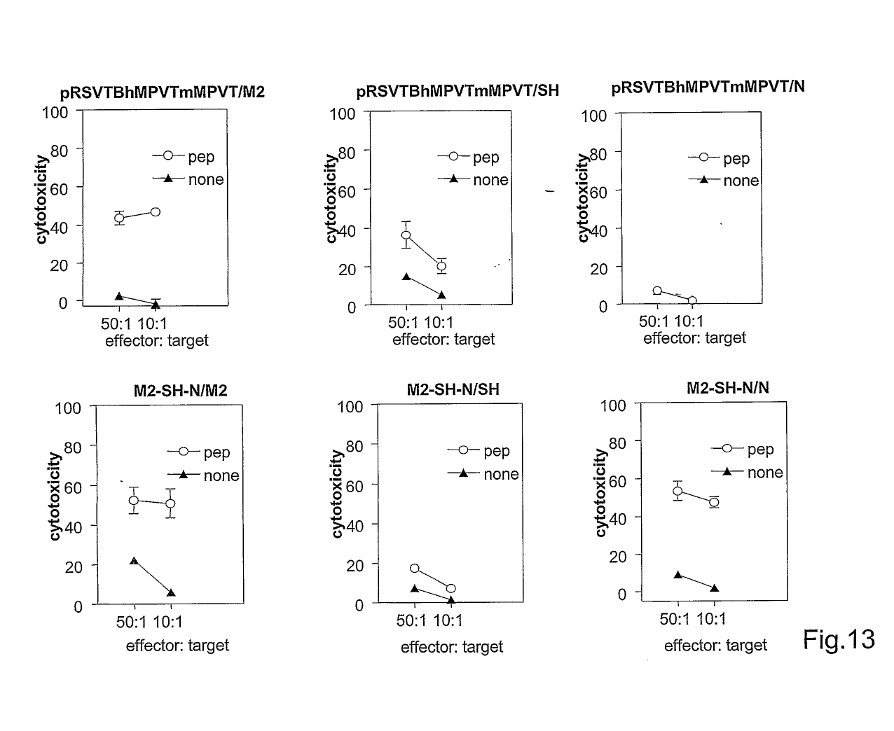

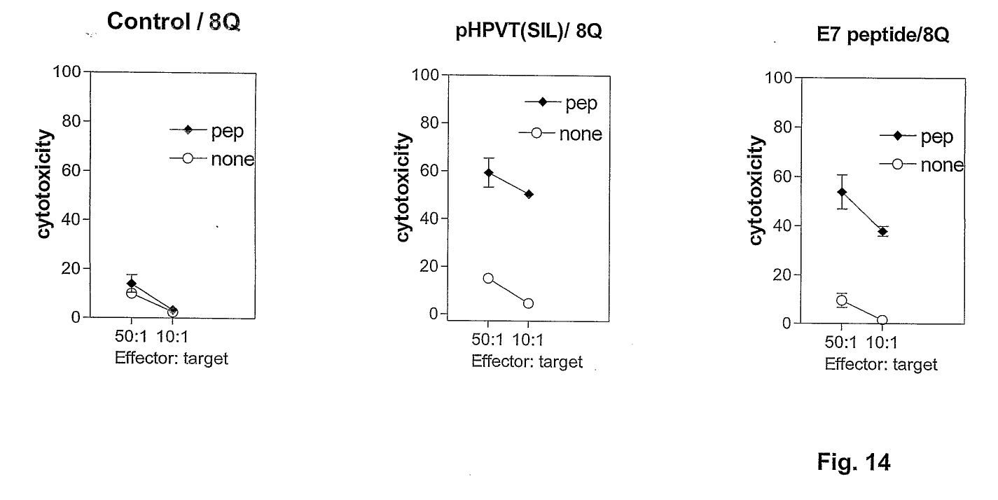

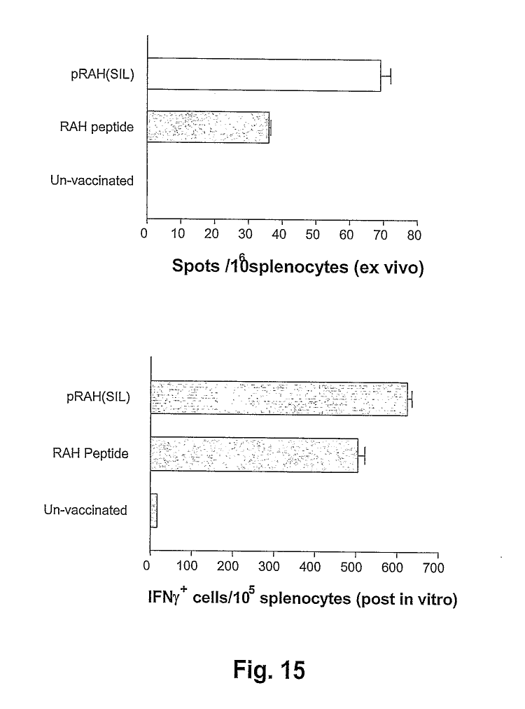

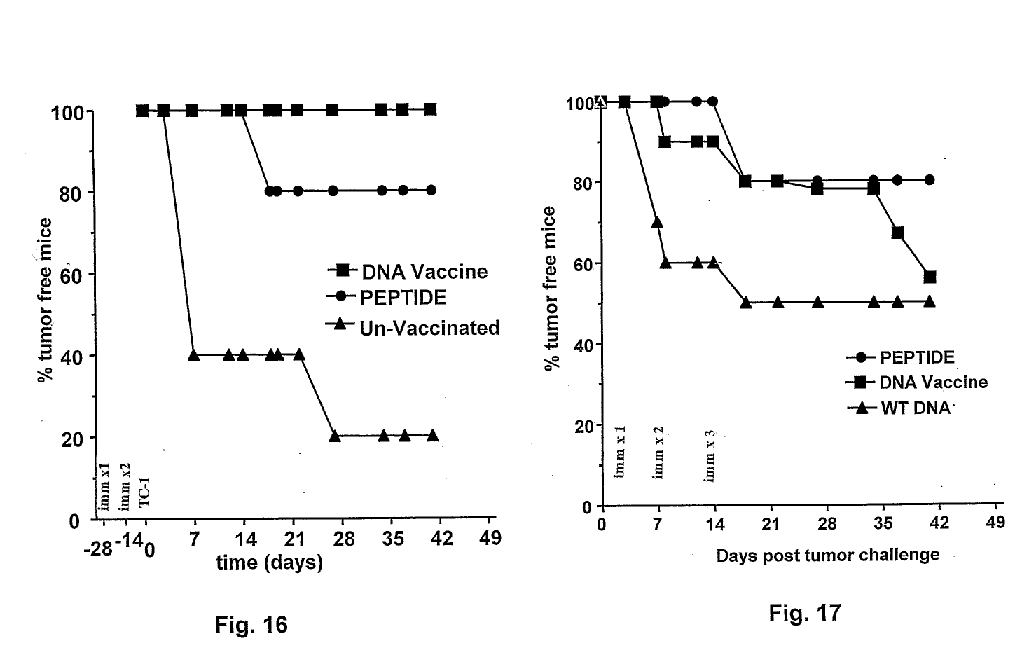

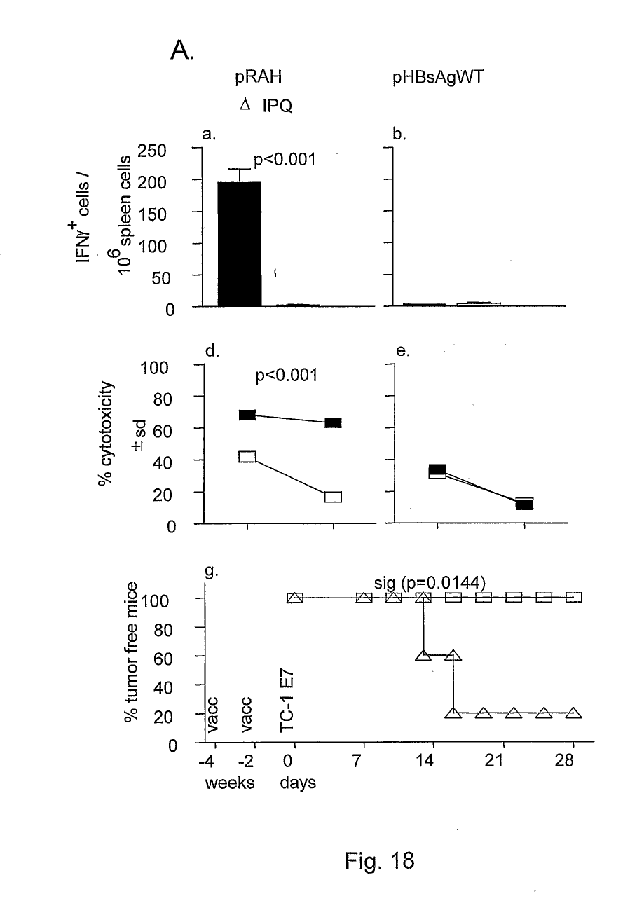

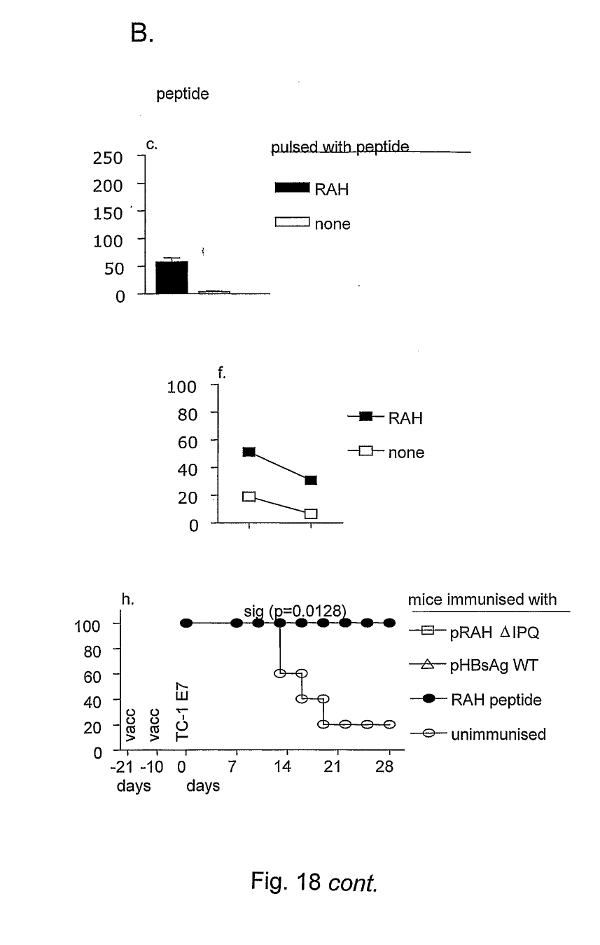

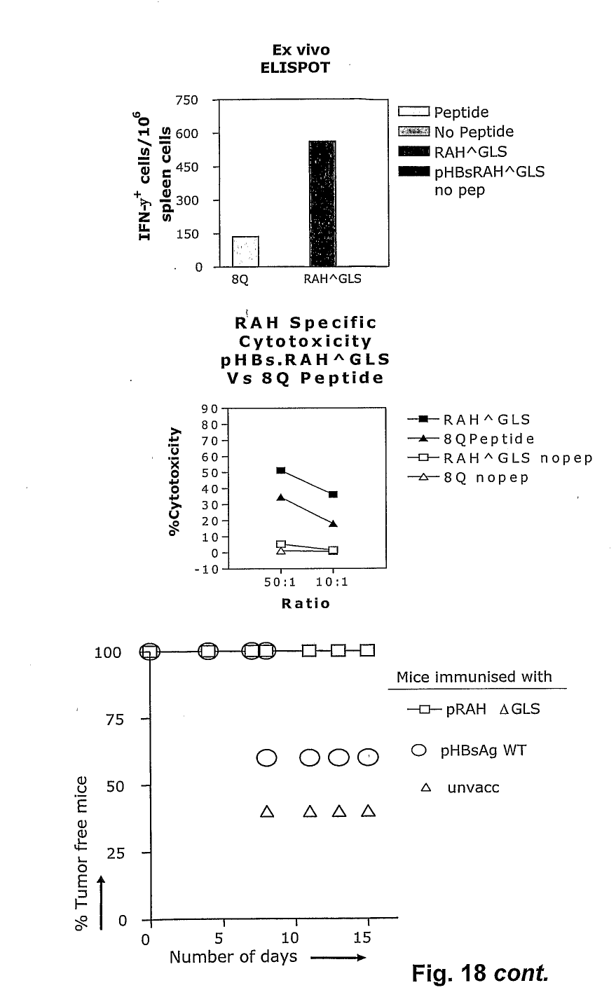

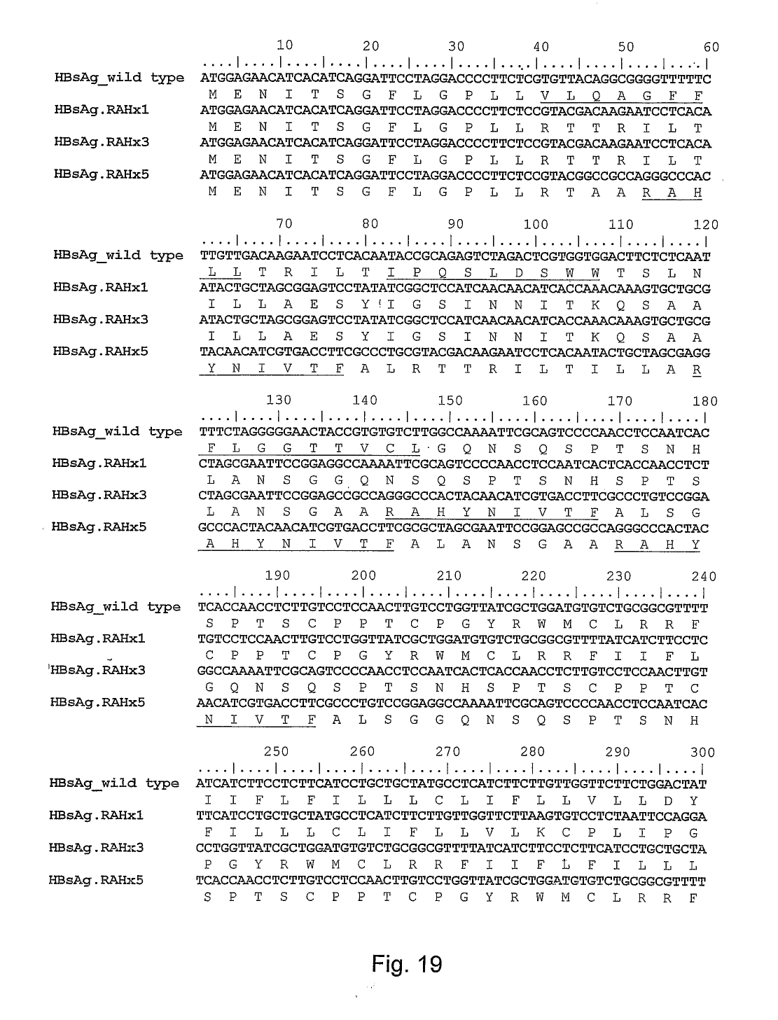

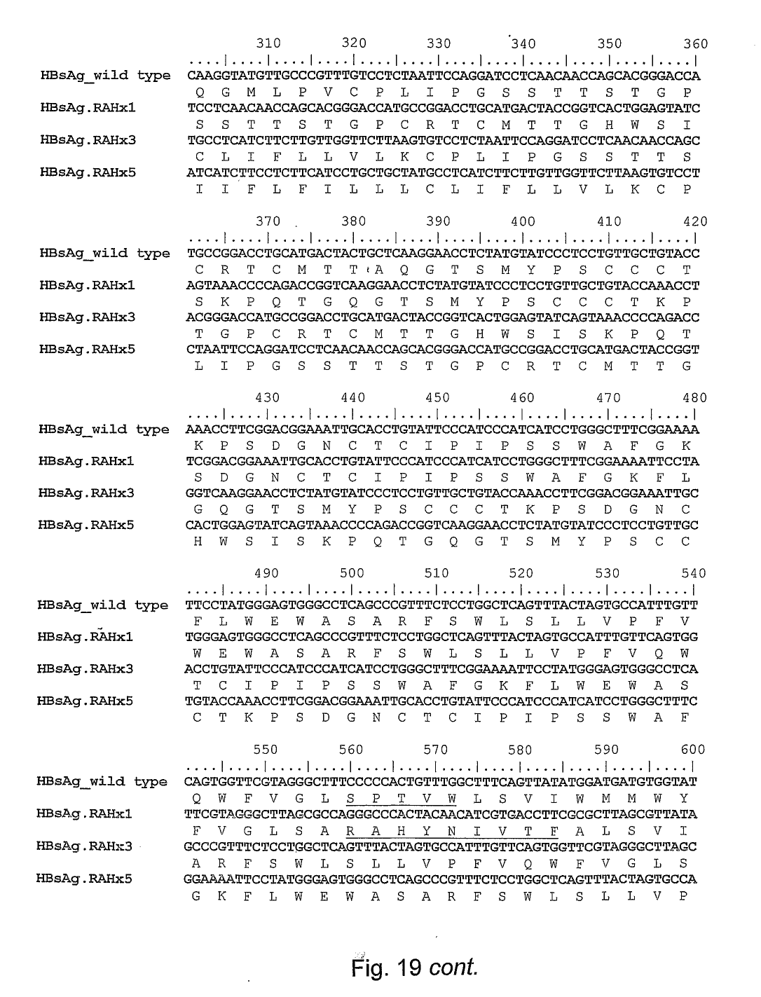

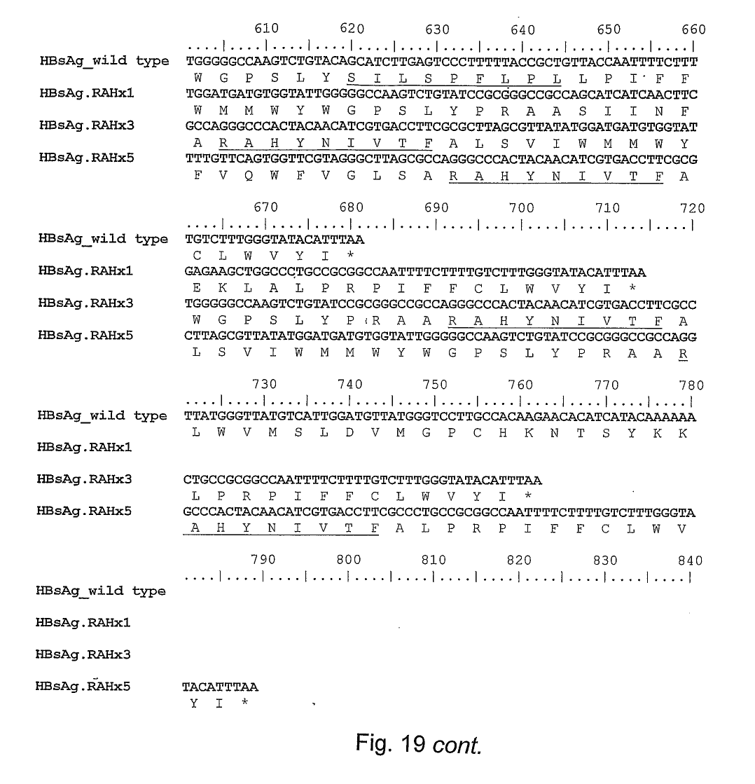

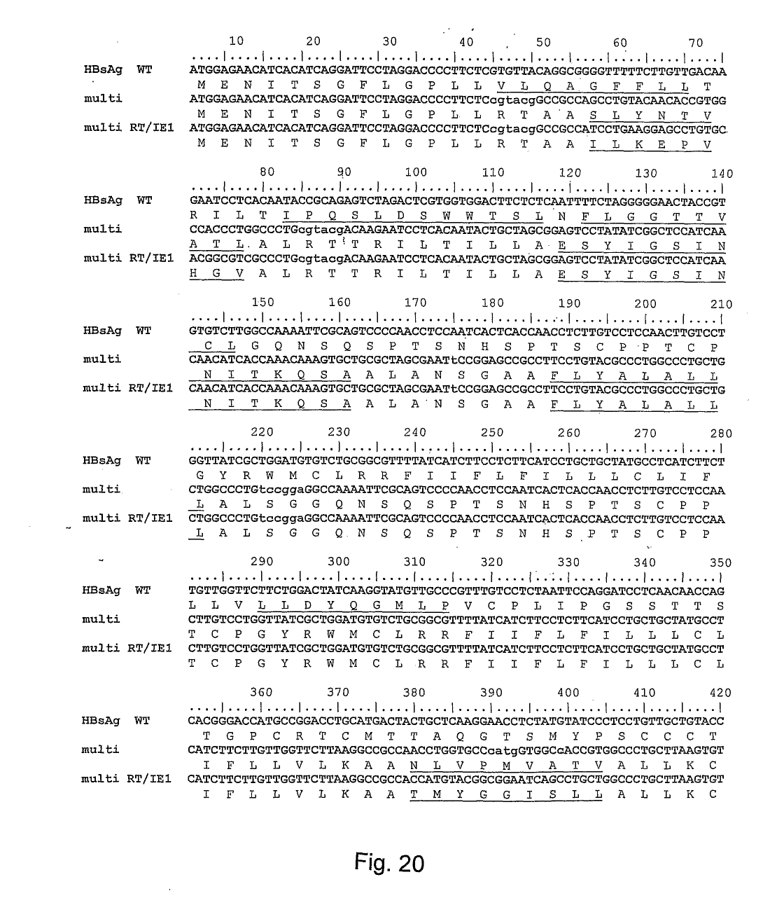

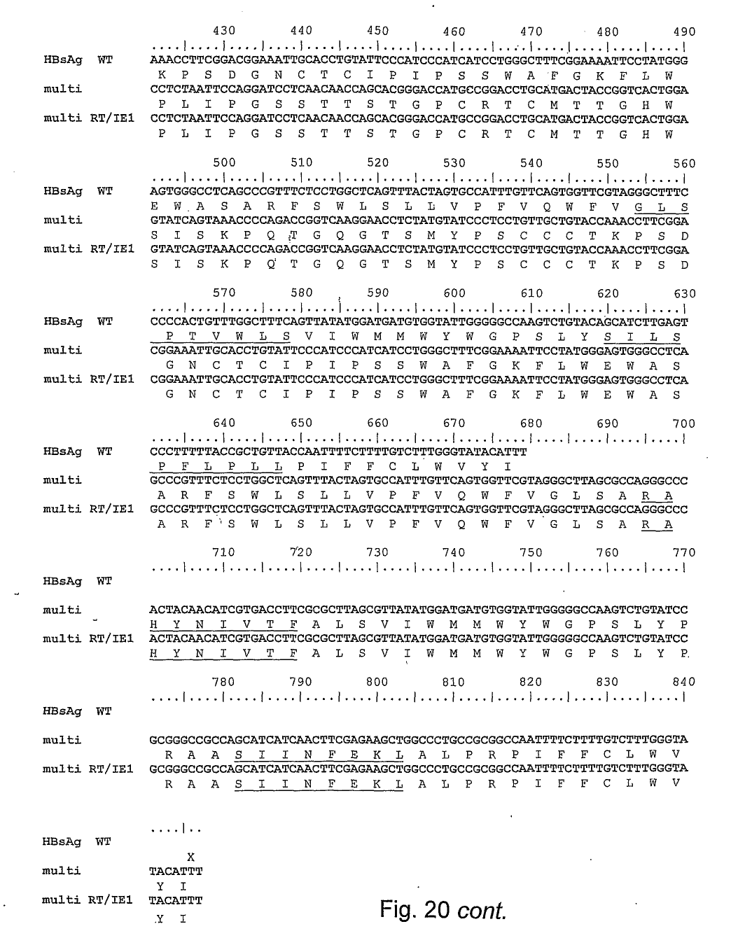

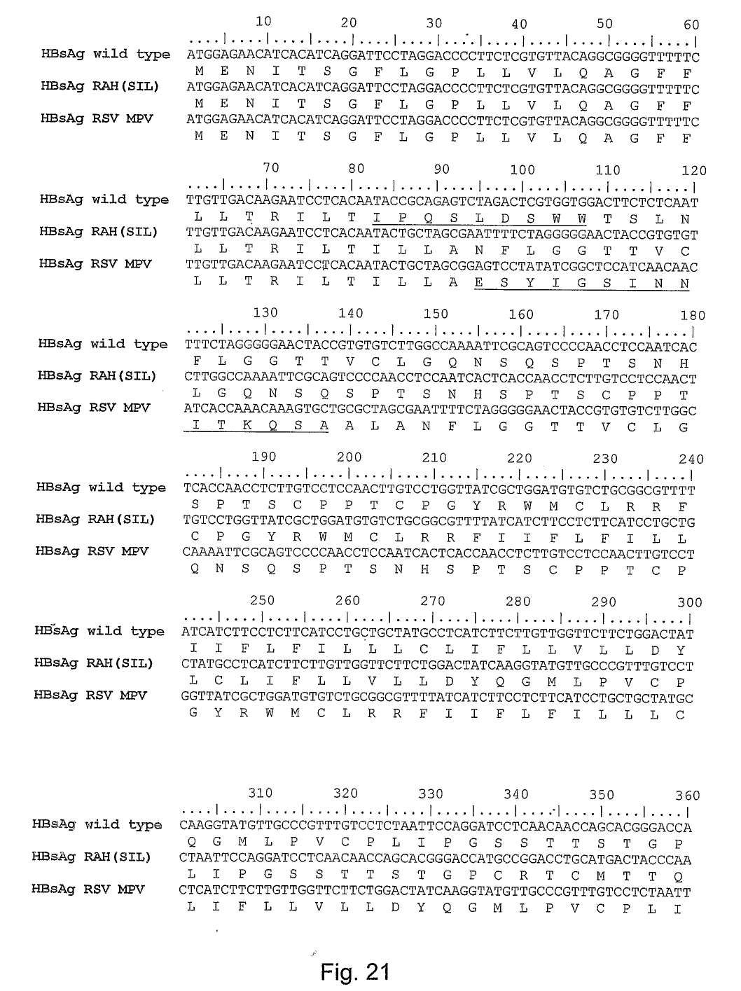

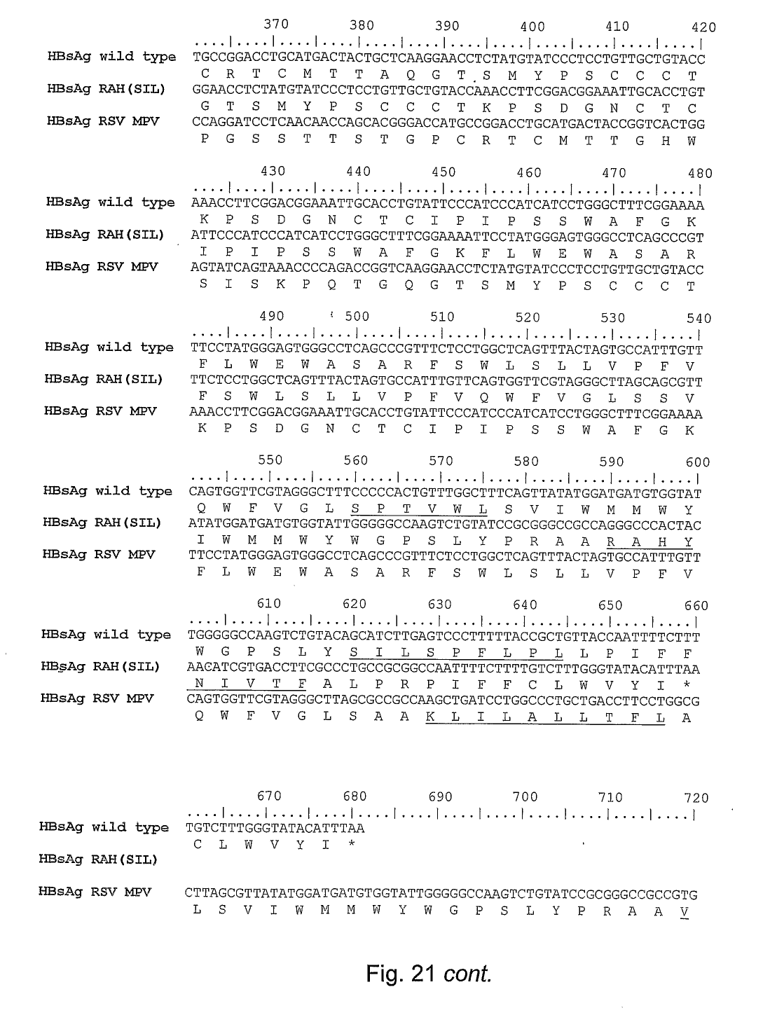

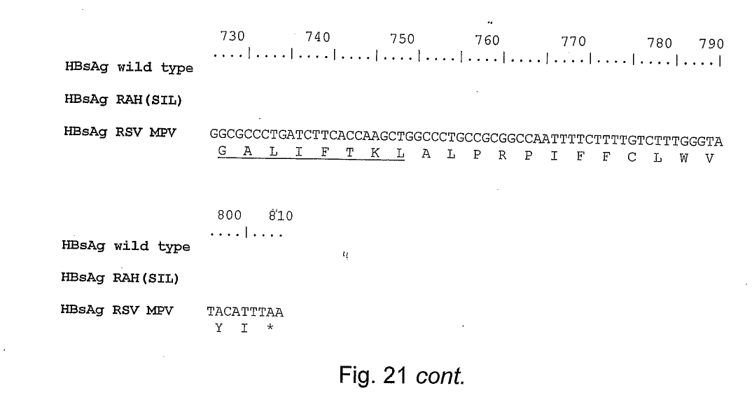

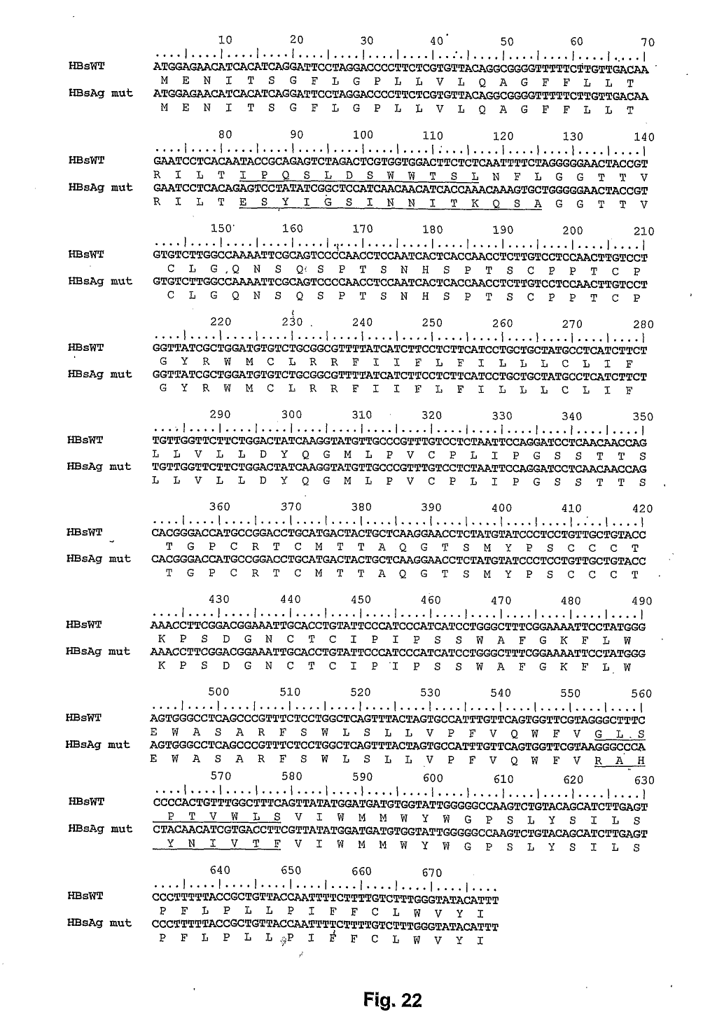

The inventors wished to determine whether rHBsAg DNA encoding the CTL epitope of RSV M2 protein in place of deleted DNA encoding the endogenous IPQ epitope, would elicit M2 directed effector T cell responses when the rHBsAg DNA was used to immunise mice. The inventors also wished to compare the M2-directed T cell response elicited by rHBsAg DNA immunisation with that elicited by immunisation with a molar excess of M2 CTL epitope peptide plus adjuvant. (Immunisation with high dose CTL epitope peptide plus adjuvant is a powerful inducer of CTL responses (Doan et al. 1998 supra). Epitope-specific interferon-gamma (IFN-γ)-secretion of ex vivo splenocytes by ELISPOT assay was quantified. Splenocytes from mice immunised with pM2₉ΔIPQ or pM2₁₅ΔIPQ DNA secreted IFN-γ when stimulated with M2₉ peptide in vitro (FIG. 3A).

While IFN-γ secretion by ex vivo splenocytes in the presence of cognate peptide is a marker of epitope-specific effector T cell function, the inventors determined whether immunisation with pM2₉ΔIPQ or pM2₁₅ΔIPQ would induce memory CTL capable of being restimulated in vitro to kill target cells displaying the M2 CTL epitope. Splenocytes from pM2₉ΔIPQ or pM2₁₅ΔIPQ DNA-immunised mice were restimulated in vitro for 6 days with M2₁₅ peptide, and reacted with H-2^{d} target cells pulsed with M2₁₅ epitope peptide. Restimulated splenocytes from both pM2₉ΔIPQ or pM2₁₅ΔIPQ immunised mice killed M2₁₅ peptide-pulsed targets with an efficiency similar to that seen in identically restimulated splenocytes from M2₁₅ peptide-plus-adjuvant immunised mice (FIG. 3B). Interestingly, killing of M2-pulsed target cells by restimulated splenocytes from pM2₉ΔIPQ or pM2₁₅ΔIPQ immunised mice was more efficient than killing of IPQ-pulsed target cells by restimulated splenocytes from pHBsAg wild-type-, or IPQ peptide+adjuvant-, immunised mice ie. killing through the endogenous IPQ epitope (FIG. 3B).

Taken together, these data indicate that replacement of DNA encoding an endogenous HBsAg CTL epitope (IPQ) with DNA encoding a CTL epitope from the RSV M2 protein to generate recombinant HBsAg DNA immunogens engenders M2-directed effector and memory CTL responses of comparable intensity to those engendered to the endogenous epitope (IPQ) by immunisation with pHBsAg wild-type DNA. Additionally, the M2-directed memory CTL response engendered by immunisation with pM2₉ΔIPQ or pM2₁₅ΔIPQ DNA is comparable to that engendered by immunisation with a molar excess of M2 peptide-plus-adjuvant.

Insertion into the HBsAg Gene of DNA Encoding the RSV M2 CTL Epitope at ΔGLS Elicits RSV M2-Directed Effector and Memory CTL Responses when the rHBsAg DNA is Used to Immunise Mice.

The inventors asked whether the strategy of deleting DNA encoding an endogenous HBsAg CTL epitope and replacing it with DNA encoding a foreign CTL epitope can be applied to sites other than IPQ within the HBsAg gene. The DNA encoding an endogenous HBsAg CTL epitope at position 348-357 (GLS) was deleted, and a BlpI unique restriction enzyme site was inserted (FIG. 1 and Table 2). DNA encoding the RSV M2₁₅ epitope was inserted at this site to derive pM2₁₅ΔGLS. The inventors asked whether immunisation with this construct would elicit M2-directed effector and memory CTL responses. Ex vivo splenocytes from mice immunised with pM2₁₅ΔGLS DNA specifically secreted IFN-γ when stimulated with M2 peptide in vitro, but not in the absence of M2 peptide (FIG. 4A).

To determine whether immunisation with pM2₁₅ΔGLS DNA would induce memory CTL capable of being restimulated in vitro to kill target cells displaying the M2 CTL epitope, splenocytes from immunised mice were restimulated in vitro for 6 days with M2 peptide, and then tested for the ability to kill target cells pulsed with M2 epitope peptide. The restimulated splenocytes killed M2-pulsed targets cells, but not unpulsed target cells (FIG. 4B). The efficiency of killing exceeded that of identically restimulated splenocytes from mice immunised with a molar excess of M2 epitope peptide-in-adjuvant (FIG. 4B).

Together with the data from FIGS. 3 and 4 show that immunisation with rHBsAg DNA in which either of two encoded endogenous HBsAg CTL epitopes (IPQ and GLS) are replaced with a foreign CTL epitope (RSV M2) elicits effector and memory CTL responses to the encoded replacement epitope.

Immunisation with rHBsAg DNA Encoding a CTL Epitope of RSV M2 Protein Partially Protects Against RSV Infection

The inventors investigated whether prior immunisation with rHBsAg DNA encoding a RSV CTL epitope would protect against RSV infection. Groups of mice were immunised with pM2₁₅ΔIPQ DNA, with pM2₁₅ΔGLS DNA, with pHBsAg wild type DNA, or with a molar excess of M₁₅ peptide+adjuvant. Three weeks later, the mice were challenged intranasally with RSV. Four days after challenge, mice were sacrificed, and lungs removed for RSV quantitation, histology and immunocytochemistry. Four of five mice immunised with wild-type HBsAg displayed detectable lung RSV, whereas only one of five mice immunised with pM2₁₅ΔGLS DNA or with pM2₁₅ΔIPQ DNA displayed detectable RSV in lungs (Table 3). One of five mice immunised with a molar excess of M₁₅ peptide+adjuvant displayed detectable RSV in lungs. Among those mice where RSV could be detected, the viral loads per lung were significantly less in mice immunised with pM2₁₅ΔGLS DNA or with pM2₁₅ΔIPQ DNA than in mice immunised with pHBsAg wild type DNA (Table 3)

The lungs of unimmunised RSV challenged mice showed severe bronchiolitis characterised by bronchiolar epithelial cell damage, extensive infiltrations of inflammatory cells, macrophages and lymphocytes (FIG. 5 (i), thin arrow), and pus formation in the inflamed bronchiole (thick arrow). This pathogenesis is consistent with severe interstitial pneumonia. Lungs from protected mice immunised with pM2₁₅ΔIPQ DNA or pM2₁₅ΔGLS DNA and challenged with RSV displayed lesser bronchiolar epithelial cell damage (FIG. 5 (ii) and (iii), thick arrows), lesser infiltration of mononuclear cells, restricted mainly to the peribronchiolar region (FIG. 5 (ii) thin arrow), with little mononuclear cell infiltration of the aveolae, and some haemorrhaging into bronchioles (FIG. 5 (iii), thin arrow). Lungs from unchallenged unimmunised mice showed little mononuclear cells infiltrate, and unimpeded alveolar spaces (FIG. 5 iv).

We immunostained lung cryosections from mice immunised with pM2₁₅ΔIPQ DNA, or with control pHBsAg wild type, or with a molar excess of M2₁₅ peptide+adjuvant, for the presence of RSV. Unprotected mice immunised with pHBsAg wild type DNA and subsequently challenged with RSV displayed intense peri- and extra-bronchiolar staining consistent with a fulminant RSV infection (FIG. 5C (i)) whereas lungs from protected mice immunised with pM2₁₅ΔIPQ DNA and subsequently RSV challenged showed much reduced staining (FIG. 5C (ii)). The reduced staining was similar to that observed in the lungs of RSV challenged protected mice which were previously immunised with M2 peptide+adjuvant (FIG. 5C (iii))

Together the data in Table 3 and FIG. 5 indicate that immunisation with rHBsAg DNA expressing a CTL epitope of the M protein of RSV gives some protection against RSV infection as measured by reduced viral load in lungs. The protection was similar to that seen in mice immunised with a molar excess of the corresponding RSV CTL epitope peptide+adjuvant. Reduced viral load was associated with a reduction in severity of lung pathology and reduction in RSV-specific immunostaining.

Immunisation with rHBsAg DNA Encoding a CTL Epitope of the HPV 16 E7 Oncoprotein Elicits E7 Directed Effector and Memory CTL Responses, and Protects Against E7 Expressing Tumour

The inventors inquired whether immunisation with rHBsAg DNA expressing a CTL epitope from a tumour associated antigen (taa) would elicit a taa-directed CTL response, and protect mice against tumour challenge. DNA encoding the H-2 D^{b} restricted RAH CTL epitope of the E7 oncoprotein of HPV16 (Table 1) was inserted into the ΔIPQ site of the HBsAg gene to create the recombinant construct pRAHΔIPQ. The inventors asked whether immunisation with pRAHΔIPQ DNA would elicit a RAH D^{b} restricted T cell response. The RAH-specific IFN-γ secreting splenocytes harvested ex vivo after two immunisations of H-2^{b} mice with pRAHΔIPQ DNA were quantified. Splenocytes incubated with RAH peptide contained a statistically significant higher number of IFN-γ secreting cells than splenocytes cultured without RAH peptide (FIG. 6A) and compared favourably with the number of IFN-γ secreting cells from mice immunised with a molar excess of RAH peptide+adjuvant. These data indicate specific in vivo priming of RAH-directed effector T cells by immunisation with pRAHΔIPQ DNA (but not pHBsAg wild-type DNA) (FIG. 6A).

The inventors asked whether immunisation with pRAHΔIPQ DNA would induce a RAH-directed memory CTL response. Mice were immunised twice with pRAHΔIPQ DNA. Control mice were immunised with pHBsAg wild-type DNA, or a molar excess of RAH peptide+adjuvant. Splenocytes from immunised mice were restimulated in vitro with RAH peptide and reacted with RAH-pulsed, IPQ-pulsed (as appropriate) or unpulsed target cells in a ⁵¹Cr-release assay. Effector cells from pRAHΔIPQ DNA immunised mice, but not pHBsAg wild-type DNA immunised mice specifically lysed target cells pulsed with RAH peptide (FIG. 6B).

The in vivo induction of RAH-directed IFN-γ secreting splenic T cells and the in vitro recall of RAH-specific memory splenic T cells following immunisation with pRAHΔIPQ DNA compared favourably with that induced by immunisation with a molar excess of RAH peptide+adjuvant (FIGS. 6A, B)

To determine whether immunisation would protect against E7-expressing tumour, groups of mice were immunised twice with pRAHΔIPQ DNA or with pHBsAg wild-type DNA, and challenged with TC-1 tumour cells two weeks after the second immunisation. Eighty percent of mice immunised with pHBsAg wild-type DNA developed tumour within 15 days. In contrast, none of the mice immunised with pRAHΔIPQ DNA developed tumour. This level of protection was consistent with that observed in a control group of mice immunised with a molar excess of RAH peptide+adjuvant (FIG. 6C)

Together, these data indicate that immunisation with DNA encoding rHBsAg containing a CTL epitope (RAH) of HPV16E7 oncoprotein protects mice against challenge with a E7-expressing tumour, and this protection is associated with RAH-directed effector and memory CTL responses.

Immunisation with rHBsAg DNA Encoding Two Copies of an Inserted Foreign Epitope does not Elicit Enhanced CTL Responses to the Inserted Epitope Compared with rHBsAg Encoding a Single Copy.

The inventors inquired whether two copies of a foreign CTL epitope, inserted at ΔIPQ and ΔGLS sites in rHBsAg respectively would give an enhanced foreign epitope-directed CTL response compared with rHBsAg containing a single copy at the ΔIPQ site or at the ΔGLS site alone. Mice were immunised with pM2₁₅ΔIPQ.ΔGLS.HWI (encoding two copies of M2₁₅ epitope and a B-cell epitope HWI) with pM2₁₅ΔIPQ.HWI (encoding one copy of M2₁₅ epitope) or with pM2₁₅ΔGLS (encoding one copy of M2₁₅ epitope) (Table 1) three times at 2 week intervals. CTL responses were examined by IFN-γ ELISPOT on ex vivo splenocytes harvested 4 weeks later, and by ⁵¹Cr-release cytoxicity on specifically restimulated splenocytes. No augmentation in the number of IFN-γ secreting splenocytes was recorded among mice immunised with rHBsAg DNA encoding two copies the M2 epitope compared with rHBsAg DNA encoding one copy (FIG. 7A). Similarly, restimulated splenocytes from mice immunised with rHBsAg DNA encoding two copies of the M2₁₅ epitope did not display enhanced killing of specific target cells when compared with restimulated splenocytes from mice immunised with rHBsAg DNA encoding a single copy of M2₁₅ (FIG. 7B). These data are consistent with the notion that provision of two copies of the M2₁₅ CTL epitope minigene within the rHBsAg DNA immunogen does not augment the CTL responses compared with provision of one copy of the minigene, under the immunisation regimen used in the experiment.

Immunisation with rHBsAg DNA Encoding Two Protective CTL Epitopes from Distinct Pathogens Elicits CTL Responses to Both Epitopes.

The inventors asked whether M2₁₅ CTL epitope of RSV and the RAH CTL epitope of HPV inserted at the ΔIPQ and ΔGLS sites respectively in the same rHBsAg molecule would elicit CTL to both epitopes. Mice were immunised with pM2₁₅ΔIPQ.RAHΔGLS.HW1 DNA (Table 2) three times at 2 week intervals, and Th1-specific cellular responses were examined by IFN-γ ELISPOT on ex vivo splenocytes harvested 4 weeks, and by ⁵¹Cr-release cytotoxicity of specifically restimulated splenocytes. The results were compared with those obtained from splenocytes from mice immunised with pM2₁₅ΔIPQ DNA or with pRAHΔGLS DNA single recombinants which contain only the M2₁₅ minigene or the RAH minigene respectively. Splenocytes from mice immunised with rHBsAg DNA encoding both epitopes (pM2₁₅ΔIPQ.RAHΔGLS. HW1) secreted IFN-γ when cultured with M2₁₅ peptide or with RAH peptide (FIG. 8A). The M2₁₅-directed response was similar in mice immunised with the double recombinant encoding both M2₁₅ and RAH minigenes or with the single recombinant encoding the M2₁₅ minigene alone (*p>0.05). The RAH-directed response was lower in mice immunised with the double recombinant compared with the single recombinant encoding the RAH-minigene alone pRAHΔGLS (**p<0.001)

Restimulated splenocytes from mice immunised with rHBsAg DNA encoding both epitopes (pM2₁₅ΔIPQ.RAHΔGLS.HW1) killed target cells pulsed with either M2₁₅ peptide, or with RAH peptide (FIG. 8B). The killing of RAH peptide pulsed targets was less than that observed with restimulated splenocytes from mice immunised with rHBsAg DNA encoding RAH minigene alone (pRAHΔGLS) (***p<0.001).

In view of the down-regulated CTL response to the RAH epitope in mice immunised with the double recombinant, we asked whether these mice were nonetheless protected against RAH-expressing tumour. Groups of mice were immunised with a single recombinant DNA encoding the RAH epitope (pRAHΔIPQ, Table 2), with pM2₁₅ΔIPQ.RAHΔGLS.HWI DNA, or with control DNA, and subsequently challenged with 8×10⁵ TC-1 tumour cells. Mice immunised with the single recombinant and mice immunised with pM2₁₅ΔIPQ.RAHΔGLS. HW1 were both fully protected against E7-expressing tumour, whereas 40-60% unvaccinated mice or mice immunised with wild-type HBsAg DNA, or rHBsAg DNA not encoding the RAH epitope, developed tumour by 8 days (FIG. 8C)

Together, these data indicate that immunisation with rHBsAg DNA encoding two foreign CTL epitopes elicits effector and memory CTL responses to both epitopes. Although the RAH-directed CTL response measured in vitro was down regulated in mice immunised with the recombinant encoding two epitopes nonetheless these mice were protected against tumour challenge, under the conditions of the experiment, as effectively as mice immunised with the recombinant encoding the RAH epitope alone.

### DISCUSSION

In the present study the inventors sought to exploit the inherent immunogenicity of HBsAg DNA as a carrier for the delivery of disease-relevant foreign CTL epitopes. The inventors believed that the simplistic approach of inserting CTL-epitope encoding sequences into the HBsAg gene at sites where restriction endonuclease sites fortuitously exist, not accounting for spatial requirements and tertiary configuration of the protein, was likely to yield unpredictable outcomes, and therefore constrained the exploitation of HBsAg to deliver inserted foreign CTL epitopes. The inventors reasoned that by deleting endogenous CTL epitopes, and replacing them with foreign CTL epitopes of similarly size and physical characteristics (hydrophobicity), they might avoid deleterious effects on the structural integrity of the recombinant HBsAg protein.

Furthermore, the inventors reasoned that substitution of proven endogenous epitopes with foreign T cell epitopes may result in an appropriate antigen processing of the foreign epitope.

The inventors elected to substitute two of the HBsAg endogenous CTL epitopes (IPQ and GLS; Table 2) with protective foreign epitopes from the M2 protein of RSV and/or the E7 protein of HPV16. In spite of the approach of replacing the IPQ or GLS endogenous epitopes with the M2 CTL epitope, the assembly and/or stability and secretion of recombinant HBsAg in vitro was reduced when compared with wild-type particles (FIG. 2). Insertion of the M2 epitope at the ΔIPQ site was associated with a ca 35-fold reduction in HBsAg protein in the supernatant of cells transfected with rHBsAg DNA, compared with insertion at the ΔGLS site. This reduction in secretion of HBsAg correlated with a reduced capacity to package hepatitis delta large antigen for secretion (FIG. 2B). The inference is that the replacement insertions interfere with the assembly or stability of structurally appropriate HBsAg particles (reflected in compromised packaging/secretion of hepatitis delta large antigen). Insertion of the 15 mer version of the M2 epitope (M2₁₅) at the IPQ site resulted in less secretion than insertion of the 9 mer version (M2₉). Therefore, the length of the inserted epitope may affect secretion. It is noteworthy however that impaired secretion of functionally intact HBsAg particles occurred even when the replacement of the endogenous epitope with foreign epitope resulted in no net change in the number of amino acid residues in the rHBsAg compared with wild-type (FIG. 1; pM2₉ ΔIPQ). This suggests the importance of specific sequence in topogenic regions essential for particle assembly.

The ability of the constructs to initiate VLP synthesis may contribute to the efficacy of the vaccine as the VLPs may be taken up by dendritic cells and hence, the DNA vaccine delivers the antigen endogenously and exogenously. However, secretion may not be essential to induce a CTL response.

Impaired particle formation vindicates DNA as the preferred approach to rHBsAg vaccination, particularly since particle formation in vivo is not an essential or major requirement for CTL induction following immunisation with HBsAg DNA (Sbai, et al. 2002, Vaccine, 20, 3137-3147).

The inventors demonstrated that delivery of inserted foreign CTL epitopes at two sites from which endogenous HBsAg CTL epitopes have been deleted elicited CTL responses comparable to or exceeding those delivered by a vast molar excess of epitope peptide+Quil A adjuvant. This latter approach induces maximal, though short-lived responses (which limits this approach for vaccination against disease), and is a ‘gold standard against which other CTL inducers may be compared.

Replacement of the DNA encoding the endogenous IPQ epitope with DNA encoding either M2₉, M2₁₅ or RAH epitopes produced recombinant HBsAg DNA immunogens which elicited CTL responses to the foreign CTL epitope better than the CTL response to the IPQ endogenous epitope which occurred following immunisation with wild-type HBsAg (FIGS. 2B and 8B). In general, the magnitude of the CTL response to a given replacement epitope in rHBsAg was more in accord with that produced by immunisation with the corresponding peptide+adjuvant than with the position at which the replacement epitope was inserted into the HBsAg molecule (FIGS. 2, 3, 8). These observations suggest that choice of sites for inserting replacement foreign epitopes within the HBsAg molecule need not be overly influenced by the magnitude of the response to the endogenous HBsAg epitope situated at that site. Therefore the sequence of the epitope may be more important than the position of the epitope (provided it is at a “CTL position”).

However, it has also been surmised that response is a function not only of primary amino acid sequence, but of the context of the epitope within the protein such that intramolecular competition for cellular processing and HLA binding influences the magnitude of the response (Perkins, et al. 1991, J. Immunol., 146, 2137-2144). It is also noteworthy that an endogenous HBsAg CTL epitope can be replaced with a foreign CTL epitope restricted through a different MHC class I haplotype, to elicit strong insert-directed CTL responses (Table 2 and FIGS. 3, 4, and 6).

The inventors demonstrated that epitope specific CTL responses induced by immunisation with rHBsAg DNA expressing the M2 RSV CTL epitope were associated with reduced proliferation of virus in the lungs of RSV challenged mice, and with amelioration in pulmonary pathology. Similarly, specific CTL responses induced by immunisation with rHBsAg DNA expressing a CTL epitope of the E7 oncoprotein of HPV16 were associated with protection against challenge with an E7 expressing tumour. The inventors also showed HBsAg recombinant for both RSV- and HPV-CTL epitopes elicited simultaneous responses to both epitopes. Together these data suggest the applicability of HBsAg as vector to deliver protective CTL responses to human disease-relevant foreign epitopes, and suggest the possibility of simultaneous protection against multiple diseases with a single polyvalent HBsAg vector. The observation that insertion of both M2₁₅ and RAH epitopes into HBsAg downregulated the CTL response to RAH, compared with a single insertion of RAH alone (FIG. 8) may indicate an immunodominance effect (at least in vitro). Nonetheless, RAH-mediated in vivo protective efficacy of HBsAg containing both foreign epitopes was still apparent (FIG. 8C). The approach of delivering a limited number of MHC class 1-restricted epitopes to target multiple diseases in humans using HBsAg DNA as a vector is feasible since a) at least 15 endogenous CTL epitopes in HBsAg are candidates for replacement (Loirat, et al. 2000J. Immunol, 165, 4748-4755; Nayersina, et al. 1993, J. Immunol., 150, 4659-4671; Wild, et al 1999, J. Immunol., 163, 1880-1887), b) in general, CTL-mediated pathogen and tumor protection is remarkably parsimonious in the number of CTL epitopes to which the responses must be directed (Rosenberg 2001, Nature, 411, 380-384; Hsu, et al. 1999, J. Gen. Virol., 80, 1401-1405) and c) a limited number of major MHC class 1 supertypes account for the majority of MHC polymorphism (Sette & Sidney 1999, Immunogenetics, 50, 201-212).

Alternatively, since administration of CTL epitopes as multiple copies in repetitive arrays is known to enhance induction of CTL response (Kruger, et al. 1999, Biol. Chem. 380, 275-276), vaccine delivery of rHBsAg recombinant for multiple copies of a single foreign epitope, each copy replacing one of the endogenous HBsAg CTL epitopes, may elicit superior responses to a single or double copy (FIG. 7) of the foreign epitope in HBsAg.

The inventors investigated extending the concept of delivery of foreign protective CTL epitopes by HBsAg vector to include simultaneous delivery of protective B cell epitopes inserted into the hydrophilic ‘a’-loop of the HBsAg molecule. However, they showed that antibody induction to an inserted B-mimotope of RSV was lost when a CTL epitopes was inserted at the site (FIG. 9). This may relate to loss of conformation which insertion at the ΔIPQ seems to bestow (cf FIG. 2), which may have particular relevance for the ‘a’-loop tertiary structure due to proximity of the IPQ site to that region. Clearly, the concept of co-delivery of B and CTL epitopes needs to be further explored using insertion sites more distal to the ‘a’-loop for CTL epitope insertion. However, proximity of B cell epitope to T epitope is not necessarily the predominant issue in determining whether or not an antibody response is obtained to the B epitope. Preservation of the structural integrity of the external ‘a’ loop where the B epitope is inserted is likely to crucial, and there are a number factors related to T epitope insertion which impact upon this, not only proximity.

While CD8 CTL responses vary in their requirement for CD4+ T cell ‘help’, it is prudent that a source of ‘help’ be included in putative CTL-inducing vaccines. That priming of CTL does not occur in CD4 knockout immunised with HBsAg suggests that CD4+ T cell ‘help’ is required (Wild, et al. 1999, supra). HBsAg per se contains Th-helper epitopes (Bryder, et al. 1999, DNA Cell Biol., 18, 219-225; Milich, 1988, Immunol. Today, 9, 380-386). Alternatively, CpG dinucleotides in bacterial DNA or synthetic oligo deoxynucleotides (ODN) prime CD4+ T-helper responses and CTL when using HBsAg DNA vaccines (Davis, et al. 1998, J. Immunol., 160, 870-876). Of further benefit is that cytokines characteristic of Th1 phenotype are produced by immunisation with plasmid HBsAg DNA (Leclerc, et al. 1997, Cell Immunol., 179, 97-106) CTL responses to HBsAg DNA vaccines can further be augmented by co-delivery of cytokine encoding plasmids, eg. IL-15, GM-CSF, IFN-β (Kwissa, et al. 2003, J. Mol. Med., 81, 91-101) or RANTES (Kim, et al. 2003, Virology 314, 84-91).

In summary, the inventors devised a strategy of deletion of DNA encoding endogenous HBsAg CTL epitopes and substitution with DNA encoding foreign CTL epitopes, to derive recombinant HBsAg immunogens which elicited insert-directed effector and memory CTL responses, associated with pathogen and tumour protective responses in vivo. The inventors demonstrated HBsAg recombinant for both RSV- and HPV-CTL epitopes elicited simultaneous responses to both epitopes. The data demonstrate the possibility of using HBsAg, which is already licensed as human vaccine, as a vector for the co-delivery of multiple disease relevant protective CTL responses.

### Example 2

### Multicopy Epitopes

FIG. 10 describes T cell responses to a chimeric HBsAg DNA construct encoding one or multiple copies of a single (tumour) CTL epitope at different sites. At lower effector:target ratio, it appears that RAHx3 and RAHx5 displayed optimal killing and also antigen-specific IFN-γ secretion. The nucleotide and amino acid sequences used in this study are shown in FIG. 19. Data demonstrate that increasing the number of CTL epitopes encoded by HBsAg shows a tendency to increase the magnitude of the CTL response in immunised mice. Preliminary data (not shown) indicates that tumour protection is enhanced also. Factors which are likely to determine outcome include
a single copy may exceed a threshold stimulus for maximal immune activation
Increasing dose of a single copy may be a substitute for multiple copies. Alternatively, a) density of epitope provided by multicopy may be important or b) processing may be limiting ie. 5 copies available from a single site may/may not be less immunogenic because of a ‘jam’ at proteosomal processing from that site, compared with 6 copies from different sites c) a single copy construct may simply have a structural conformation more conducive to particle formation and therefore optimal immunogenicity

Derivation of a construct containing six protective CTL epitopes from different diseases RSV, HPV, EBV, HIV, CMV (and OVA) is also contemplated using constructs with the sequences in FIG. 20.

### Example 3

### Effect of Epitope Insertion Site

FIGS. 11 to 13 describe data obtained from constructs containing protective CTL epitopes from RSV and hMPV which elicits simultaneous CTL responses to both, and thus is a potential vaccine for paediatric respiratory disease in which RSV and hMPV are major pathogens.

The nucleotide and amino acid sequences used in this study are shown in FIG. 21.

FIGS. 14 to 17 demonstrate that an epitope inserted at epitope site SIL will elicit CTL responses when that site alone is used for insertion, but will not elicit CTL responses when that site is one among several other sites at which different CTL epitopes are inserted (HG, SIL site).

Other data have demonstrated that a particular site inserted (M2ΔIPQ) may be immunodominant over a CTL epitope inserted at another site (RAHΔGLS) (See FIG. 8).

Choice of which endogenous CTL epitope to replace with foreign CTL epitope can be guided by the following principals. I) Be wary of immunodominance exerted by the IPQ site, or immunodominant foreign epitopes inserted at the IPQ site, if further epitopes are to be inserted downstream., 3) insertions at SIL don't work well in combination with other epitope insertions elsewhere. The rules governing these observations are hitherto not defined, but presumably relate in part to maintenance of tertiary structure by the translate HBsAg protein.

It is also apparent from FIGS. 14 to 18 that a tumour CTL epitope (RAH) inserted at any one of three site (IPQ, GLS and SIL) will elicit CTL and tumour prevention with more-or-less similar efficacy. At least one site (SIL) is protective against established tumours.

### Example 4

### Recombinant Approaches to Optimisation of VLP Production

The present inventors have created have created HBsAg in which endogenous CTL epitopes were replaced with foreign epitopes by site-directed mutagenesis rather than by utilizing enzyme site insertion. Examples of such sequences are shown in FIG. 22 This allows for more faithful replacement (ie. no enzyme half-sites or ‘fillers’). Comparison of ability to form VLPs by constructs derived using the two strategies is underway.

It is also envisaged that codon-optimisation of foreign epitope sequences may also facilitate expression and immunogenicity of HBsAg constructs. It is also contemplated that expression may be facilitated, improved or optimized in yeast.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, scientific and patent literature described in this specification are incorporated herein by reference in their entirety.

TABLE 1

| | DESIGNATION | PROTEIN | RESIDUE | SEQUENCE | REFERENCE |
|---|---|---|---|---|---|
| CTL Epitopes | IPQ | HBsAg | 202-213 | IPQSLDSWWTSL | Ishikawa et al., 1998. |
| | GLS | HBsAg | 348-357 | GLSPTVWLS | Loirat et al., 2000. |
| | FLL | HBsAg | 183-191 | FLLTRILTI | Loirat et al., 200 |
| | M2₉ | RSV M2 | 82-90 | SYIGSINNI | Kulkarui et al., 1995. |
| | M2₁₅ | RSV M2 | 81-95 | ESYIGSINNITKQSA | Kulkarui et al., 1995. |
| | RAH | HPV16 E7 | 49-57 | RAHYNIVTF | Faltkamp et al., 1993. |
| B Mimotope | HW1 | RSV F | n/s | HWSISKPQ | Chargelegue et al., |
| | | | | | 1998. |

TABLE 2

| Deleted | | Replaced with | | Net size | B-epitope | |
|---|---|---|---|---|---|---|
| HBsAg CTL | | foreign CTL | | change | insertion | |
| epitope (size) | MHC restriction | epitope (size) | MHC restriction | (aa)¹ | into ‘a’ loop | Construct name |
| IPQ (12 mer) | H-2L^{d} | M2₉ (9 mer) | H-2K^{d} | 0 | — | pM2₉ ΔIPQ |
| IPQ (12 mer) | H-2L^{d} | M2₁₅ (15 mer) | H-2K^{d} | +6 | — | pM2₁₅ ΔIPQ |
| IPQ (12 mer) | H-2L^{d} | RAH (9 mer) | H-2D^{b} | 0 | — | pRAH ΔIPQ |
| GLS (9 mer) | HLA A*0201 | M2₁₅ (15 mer) | H-2K^{d} | +9 | — | pM2₁₅ ΔGLS |
| GLS (9 mer) | HLA A*0201 | RAH (9 mer) | H-2D^{b} | 0 | — | pRAH ΔGLS |
| IPQ and GLS | H-2L^{d} and HLA A*0201 | M2₁₅ and M2₁₅ | H-2K^{d} | +6, +9 | — | pM2₁₅ ΔIPQ.ΔGLS |
| IPQ and GLS | H-2L^{d} and HLA A*0201 | M2₁₅ and RAH | H-2K^{d} and H-2D^{b} | +6, 0 | — | pM2₁₅ ΔIPQ.RAHΔGLS |
| IPQ and GLS | H-2L^{d} and HLA A*0201 | M2₁₅ and RAH | H-2K^{d} and H-2D^{b} | +6, 0 | HW1 | pM2₁₅ΔIPQ.RAHΔGLS.HW1² |
| — | n/a | — | n/a | | HWI | pHW1 |

TABLE 3

| | | Number of | Mean RSV |
|---|---|---|---|
| | | mice (%) with | load per ½-lung |
| Mouse | | detectable^{1.} | in mice with |
| group | Immunisation | RSV in lungs. | detectable infection. |
| 1 | pM2₁₅ Δ IPQ DNA | 1/5 (20)* | 2.2 × 10³** |
| 2 | pM2₁₅ Δ GLS DNA | 1/5 (20)* | 2.0 × 10³** |
| 3 | M2₁₅ peptide + adjuvant | 1/5 (20) | 1.0 × 10³ |
| 4 | pHBsAg W/T DNA | 4/5 (80)* | 8.0 × 10³** |
| | | | 6.0 × 10³** |
| | | | 7.0 × 10³** |
| | | | 7.0 × 10³** |

TABLE 4

| Protein and | | | |
|---|---|---|---|
| position | Peptide | Protein and position | Peptide |
| Core, 35-44 | YLLPRRGPRL | Core, 131-140 | ADLMGYIPLV |
| E1, 257-266 | QLRRIDLLV | E2, 401-411 | SLLAPGAKQNV |
| NS3, 1073-81 | CINGVCWTV | NS3, 1406-15 | KLVALGINAV |
| NS4, 1789-97 | SLMAFTAAV | NS4, 1807-16 | LLFNILGGWV |
| NS4B, 1851-59 | ILAGYGAGV | NS5, 2252-60 | ILDSFDPLV |
| NS5B, 2578-87 | RLIVFPDLGV | NS5B, 2727-35 | GLQDCTMLV |

TABLE 5

| | Native Epitope | Foreign Epitope | Source |
|---|---|---|---|
| Designation | Sequence | Sequence | Protein |
| IPQ | IPQSLDSWWTSL | Not applicable | HBsAg |
| GLS | GLSPTVWLS | Not applicable | HBsAg |
| FLL | FLLTRILTI | Not applicable | HBsAg |
| VLQ | VLQAGFFLL | Not applicable | HBsAg |
| FLG | FLGGTTVCL | Not applicable | HBsAg |
| LLD | LLDYQGMLP | Not applicable | HBsAg |
| SIL | SILSPFLPLL | Not applicable | HBsAg |
| M2₉ΔIPQ | IPQSLDSWWTSL | SYIGSINNI | RSV M2 |
| M2₁₅ΔIPQ | IPQSLDSWWTSL | ESYIGSINNITKQSA | RSV M2 |
| RAHΔIPQ | IPQSLDSWWTSL | RAHYNIVTF | HPV 16 E7 |
| RAHΔGLS | GLSPTVWLS | RAHYNIVTF | HPV 16 E7 |
| M2₁₅ΔGLS | GLSPTVWLS | ESYIGSINNITKQSA | RSV M2 |
| M2₁₅ΔIPQ.ΔGLS.HWI | IPQSLDSWWTSL | ESYIGSINNITKQSA | RSV M2 |
| | GLSPTVWLS | ESYIGSINNITKQSA | RSV M2 |
| | not applicable | HWSISKPQ | RSV F |
| M2₁₅ΔIPQ.RAHΔGLS | IPQSLDSWWTSL | ESYIGSINNITKQSA | RSV M2 |
| | GLSPTVWLS | RAHYNIVTF | HPV 16 E7 |
| M2₁₅ΔIPQ.RAHΔGLS.HW1 | IPQSLDSWWTSL | ESYIGSINNITKQSA | RSV M2 |
| | GLSPTVWLS | RAHYNIVTF | HPV 16 E7 |
| | not applicable | HWSISKPQ | RSV F |
| HBsAg.RAHx1 | GLSPTVWLS | RAHYNIVTF | HPV 16 E7 |
| HBsAg.RAHx3 | GLSPTVWLS | RAHYNIVTF | HPV 16 E7 |
| | SILSPFLPL | RAHYNIVTF | |
| | FLGGTTVCL | RAHYNIVTF | |
| HBsAg.RAHx5 | GLSPTVWLS | RAHYNIVTF | HPV 16 E7 |
| | SILSPFLPL | RAHYNIVTF | |
| | FLGGTTVCL | RAHYNIVTF | |
| | IPQSLDSWW | RAHYNIVTF | |
| | VLQAGFFLL | RAHYNIVTF | |
| HBsAg Multi | VLQAGFFLL | SLYNTVATL | HIVgag p17 |
| | IPQSLDSWWTSL | ESYIGSINNITKQSA | RSV M2 |
| | FLGGTTVCL | FLYALALLL | EBV LMP E1 |
| | LLDYQGMLP | NLVPWWRTV | CMV pp65 |
| | GLSPTVWLS | RAHYNIVTF | HPV 16 E7 |
| | SILSPFLPLL | SINNFEKL | OVA |
| HBsAg Multi RT/IE1 | VLQAGFFLL | ILKEPVHGV | HIV RT |
| | IPQSLDSWWTSL | ESYIGSINNITKQSA | RSV M2 |
| | FLGGTTVCL | FLYALALLL | EBV LMP E1 |
| | LLDYQGMLP | TMYGGISLL | CMV IE1 |
| | GLSPTVWLS | RAHYNIVTF | HPV 16 E7 |
| | SILSPFLPLL | SINNFEKL | OVA |
| HBsAg RAH (SIL) | SILSPFLPL | RAHYNIVTF | HPV 16 E7 |
| HBsAg RSV MPV | IPQSLDSWW | ESYIGSINNITKQSA | RSV M2 |
| | GLSPTVWLS | KLILALLTFL | hMPV SH |
| | SILSPFLPL | VGALIFTKL | mMPV N |
| HBsAg mut | IPQSLDSWWTSL | ESYIGSINNITKQSA | RSV M2 |
| | GLSPTVWLS | RAHYNIVTF | HPV 16 E7 |

1. An isolated protein comprising an HBsAg amino acid sequence wherein one or more immunogenic T cell epitopes of said HBsAg are respectively substituted with one or more immunogenic T cell epitopes of one or more proteins other than HBsAg.

2. The isolated protein of claim 1 wherein said T cell epitope is a CTL epitope.

3. The isolated protein of claim 2 wherein the one or more CTL epitopes of HBsAg that are substituted comprise an amino acid sequence selected from the group consisting of SEQ ID NO: 1, SEQ ID NO: 2, SEQ ID NO: 3, SEQ ID NO: 21, SEQ ID NO: 20, SEQ ID NO: 22 and SEQ ID NO: 27.

4. The isolated protein of claim 3 wherein the CTL epitope of HBsAg that is substituted comprises an amino acid sequence as set forth in SEQ ID NO: 27 only.

5. The isolated protein of claim 1 wherein said one or more proteins other than HBsAg are derived from a pathogen or is a tumour-associated antigen.

6. The isolated protein of claim 2 wherein the CTL epitope of one or more proteins other than HBsAg comprises an amino acid sequence selected from the group consisting of SEQ ID NO: 49, SEQ ID NO: 50, SEQ ID NO: 51, SEQ ID NO: 52, SEQ ID NO: 5 and SEQ ID NO: 6.

7. The isolated protein of claim 1 further comprising one or more B cell epitopes of one or more proteins other than HBsAg.

8. The isolated protein of claim 7, wherein the one or more B cell epitopes is a mimotope.

9. The isolated protein of claim 8, wherein the mimotope comprises an amino acid sequence as set forth in SEQ ID NO: 7.

10. The isolated protein of claim 7, wherein the one or more B cell epitopes of one or more proteins other than HBsAg is are inserted within an a-determinant region of HBsAg.

11. (canceled)

12. The isolated protein of claim 5, wherein said pathogen is selected from the group consisting of a bacterium, a parasite, a virus and a protozoan.

13. The isolated protein of claim 12, wherein said pathogen is selected from the group consisting of human papillomavirus, respiratory syncytial virus, human immunodeficiency virus, cytomegalovirus, Epstein Barr virus, rotavirus, hepatitis C virus, hepatitis B virus, Plasmodium falciparum, influenza virus, Mycobacterium tuberculosis, measles virus, parainfluenza virus, and human metapneumovirus.

14. The isolated protein of claim 13, wherein said pathogen is respiratory syncytial virus.

15. The isolated protein of claim 13, wherein said pathogen is human papillomavirus.

16. The isolated protein of claim 5 wherein the one or more proteins other than HBsAg may be one or more tumour associated antigens.

17. An isolated nucleic acid encoding the isolated protein of claim 1.

18. The isolated nucleic acid of claim 17, wherein the isolated nucleic acid is DNA.

19. An expression construct comprising the isolated nucleic acid of claim 17 operably-linked or connected to one or more regulatory sequences in an expression vector.

20. The expression construct of claim 19, wherein the regulatory sequences are capable of operation in a eukaryotic system.

21. A host cell comprising one or more of the expression constructs of claim 19.

22. The host cell of claim 21, which is capable of producing a virus-like particle.

23. The host cell of claim 22, wherein the host cell is of eukaryotic origin.

24. The host cell of claim 23, wherein the host cell is a mammalian cell.

25. The host cell of claim 24, wherein the host cell is a HuH-7 cell.

26. The host cell of claim 23, wherein the host cell is a yeast cell.

27. A virus-like particle comprising a plurality of isolated proteins according to claim 1.

28. The virus-like particle of claim 27, comprising an isolated nucleic acid encoding an isolated protein comprising an HBsAg amino acid sequence wherein one or more immunogenic T cell epitopes of said HBsAg are respectively substituted with one or more immunogenic T cell epitopes of one or more proteins other than HBsAg.

29. A method of producing an isolated nucleic acid including the step of substituting each of one or more nucleotide sequences of an isolated nucleic acid encoding one or more HBsAg immunogenic epitopes with a nucleotide sequence encoding one or more immunogenic epitopes of one or more proteins other than HBsAg.

30. A method of producing a virus-like particle comprising the steps of
(i) introducing one or more isolated nucleic acid of claim 17 into a cell which is capable of producing a virus-like particle;
(ii) culturing said cell under conditions that facilitate production of said virus-like particle; and
(iii) isolating said virus-like particle.

31. A virus-like particle produced according to the method of claim 30.

32. A pharmaceutical composition comprising an immunogenic agent selected from the group consisting of an isolated protein of claim 1, an isolated nucleic acid encoding the protein of claim 1, and a virus-like particle comprising a plurality of isolated proteins according to claim 1, and a pharmaceutically acceptable carrier, diluent or excipient.

33. The pharmaceutical composition of claim 32, wherein the pharmaceutical composition is an immunotherapeutic composition.

34. The pharmaceutical composition of claim 33, which is a vaccine.

35. The pharmaceutical composition of claim 32, wherein the pharmaceutical composition is capable of treating or preventing a disorder caused by pathogens selected from the group consisting of human papillomavirus, respiratory syncytial virus, human immunodeficiency virus, cytomegalovirus, Epstein Barr virus, rotavirus, hepatitis C virus, hepatitis B virus, Plasmodium falciparum, influenza virus, Mycobacterium tuberculosis, measles virus, parainfluenza virus and human metapneumovirus.

36. The pharmaceutical composition of claim 32, wherein the pharmaceutical composition is capable of treating or preventing cancer.

37. A method of treating an animal including the step of administering the pharmaceutical composition of claim 32 to said animal to thereby modulate an immune response in said animal.

38. The method of claim 37, wherein said animal is a mammal.

39. The method of claim 38, wherein said mammal is a human.

40. The method of claim 37, wherein the animal is prophylactically or therapeutically treated for a disease or disorder caused by pathogens selected from the group consisting of human papillomavirus, respiratory syncytial virus, human immunodeficiency virus, cytomegalovirus, Epstein Barr virus, rotavirus, hepatitis C virus, hepatitis B virus, Plasmodium falciparum, influenza virus, Mycobacterium tuberculosis, parainfluenza virus, measles virus and human metapneumovirus.

41. The method of claim 37, wherein the animal is prophylactically or therapeutically treated for cancer.

42. A method of immunizing an animal including the step of administering the pharmaceutical composition of claim 32 to said animal to induce an immune response in said animal.

43. The method of claim 42, wherein said animal is a mammal.

44. The method of claim 43, wherein said mammal is a human.

45. The method of claim 42, wherein the animal is prophylactically or therapeutically treated for a disease or disorder caused by pathogens selected from the group consisting of human papillomavirus, respiratory syncytial virus, human immunodeficiency virus, cytomegalovirus, Epstein Barr virus, rotavirus, hepatitis C virus, hepatitis B virus, Plasmodium falciparum, influenza virus, Mycobacterium tuberculosis, measles virus, parainfluenza virus and human metapneumovirus.

46. The method of claim 42, wherein the animal is prophylactically or therapeutically treated for cancer.

47. The isolated protein of claim 1, wherein the one or more immunogenic T cell epitopes of one or more proteins other than HBsAg are a plurality of epitopes of one or more proteins other than HBsAg.

48. The isolated protein of claim 7, wherein one or more B cell epitopes of said HBsAg are respectively substituted with one or more B cell epitopes of one or more proteins other than HBsAg.

49. The isolated protein of claim 1, wherein said isolated protein comprises an amino acid sequence selected from the group consisting of SEQ ID NO: 32. SEQ ID NO: 33. SEQ ID NO: 34, SEQ ID NO: 35, SEQ ID NO: 36, SEQ ID NO: 37, SEQ ID NO: 38, and SEQ ID NO: 39.

50. The isolated nucleic acid of claim 17, wherein said isolated nucleic acid comprises a nucleotide sequence selected from the group consisting SEQ ID NO: 41, SEQ ID NO: 42, SEQ ID NO: 43, SEQ ID NO: 44, SEQ ID NO: 45, SEQ ID NO: 46; SEQ ID NO: 47 and SEQ ID NO: 48.
